(19) United States
(12) Patent Application Publication
(10) Pub. No.: US 2024/0262829 A1
(43) Pub. Date: Aug. 8, 2024

(54) PEPTIDE NUCLEIC ACIDS, SYNTHESIS, AND USES THEREOF

(71) Applicant: Oncogenuity, Inc., Bay Harbor Islands, FL (US)

(72) Inventor: Jeffrey Rothman, Bay Harbor Islands, FL (US)

(21) Appl. No.: 18/556,628

(22) PCT Filed: Apr. 20, 2022
(86) PCT No.: PCT/US2022/025513

§ 371 Date: Oct. 20, 2023

Related U.S. Application Data
(60) Provisional application No. 63/177,815, filed on Apr. 21, 2021.

(51) Int. Cl. C07D 473/18; C07D 403/12; C07D 405/12; C07D 473/34; C07D 487/04; C07D 491/048

(52) U.S. Cl. CPC C07D 473/18; C07D 403/12; C07D 405/12; C07D 473/34; C07D 487/04; C07D 491/048

(57) ABSTRACT

The present disclosure provides peptide nucleic acids (PNAs) including cyclic structural moieties such as tetrahydrofuran, pyrrolidinium, pyrrolidine, or N-methyl pyrrolidine, which have surprisingly improved the water solubility and binding affinity of PNA oligomers to ribose-phosphate nucleic acid oligomers. Pharmaceutical compositions including the disclosed PNAs, synthetic methods thereof, and methods of use thereof are also disclosed.

### CROSS-REFERENCE TO RELATED APPLICATIONS

This application claims benefit of priority under 35 U.S.C. § 119(e) of U.S. Ser. No. 63/177,815, filed Apr. 21, 2021, the entire content of which is incorporated herein by reference in its entirety.

### BACKGROUND OF THE INVENTION

### Field of the Invention

The present invention relates generally to the field of peptide nucleic acid derivatives, pharmaceutical compositions, and methods of using the compounds and compositions containing them. The present disclosure specifically relates to peptide nucleic acid derivatives chemically modified to improve solubility, cell penetration and affinity for nucleic acid.

### Background Information

Peptide nucleic acids (PNAs) are synthetic polymers with similarities to DNA and RNA. PNAs are DNA/RNA analogs in which the sugar-phosphate backbone (composed of deoxyribose and ribose sugar backbones, respectively) is replaced by repeating N-(2-aminoethyl)-glycine units that are linked by peptide bonds. The PNA backbone does not contain any charged phosphate group which provides PNAs with a neutral backbone due to the absence of electrostatic repulsion. PNAs include natural nucleobases (purines and pyrimidines), capable of base pairing through classical Watson-Crick base pairing. The combination of the Watson-Crick hydrogen bonding scheme with the absence of electrostatic repulsion provides PNA with remarkable hybridization and stability towards complementary oligonucleotides (DNA and/or RNA). Furthermore, pyrimidine and purine bases are linked to the PNA backbone by carbonyl groups and methylene bridges. PNA backbones contain no charged phosphate groups, therefore, due to a lack of electrostatic repulsion, binding between PNA sequences and DNA (or RNA) strands is stronger than binding between two DNA (or RNA) strands. Because of the higher binding strength. PNA oligomers longer than 20-25 bases are usually not necessary. Increasing the length of PNA strands could reduce specificity for target DNA (or RNA) sequences. A PNA/DNA mismatch has greater instability than a DNA/DNA mismatch. PNAs exhibit greater specificity than DNA when binding to complementary sequences. The increased stability can be translated into a higher thermal stability as compared to natural DNA/DNA double helix of the same length, and a lack of effect of high ionic strength medium. Additionally, because enzymes are substrate specific, the recognition of PNA neutral backbone is not easy by either nucleases or proteases, making them potentially resistant to enzymatic degradation and providing them stability over wide pH range.

PNAs hybridize to complementary DNA or RNA in a sequence-dependent manner, in either parallel or antiparallel manner. However, the antiparallel binding is favored over the parallel one. The stability of the PNA/DNA duplexes is highly sensitive to the existence of a single mismatched base pair making PNA great candidate to target specific sequences, such as mutated sequences for example. PNAs can inhibit transcription and translation of genes by tight binding to DNA or mRNA. PNA-mediated inhibition of gene transcription is mainly due to the formation of strand invaded complexes or strand displacement in a DNA target. PNA-mediated inhibition of gene transcription is mainly due to the formation of PNA/RNA complexes, independently of the RNA secondary structure. Studies on the mechanisms of antisense activity have demonstrated that PNAs inhibit expression differently than antisense oligonucleotides acting through RNase-H mediated degradation of the mRNA-oligonucleotide hybrid. Since PNAs are not substrates for RNAse, their antisense effect acts through steric interference of either RNA processing, transport into cytoplasm or translation, caused by binding to the mRNA.

Cancer is a generic term for a large group of diseases involving abnormal cell growth with the potential to invade or spread to other parts of the body, and that can affect any part of the body. Cancer is the second leading cause of death globally; in 2020, about 19.3 million people were newly diagnosed with cancer, and it caused about 9.9 million deaths. The most common types of cancer are lung cancer, breast cancer, prostate cancer, colorectal cancer, stomach cancer, cervical cancer, and skin cancer. Cancer arises from the transformation of normal cells into tumor cells in a multistage process that generally progresses from a pre-cancerous lesion to a malignant tumor. These changes are the result of the interaction between a person's genetic factors and external agents, including physical carcinogens (such as ultraviolet and ionizing radiation), chemical carcinogens (such as asbestos, components of tobacco smoke, aflatoxin (a food contaminant), and arsenic (a drinking water contaminant)), and biological carcinogens, such as infections from certain viruses, bacteria, or parasites.

Besides major advances in both cancer detection and cancer treatment, through the development of early diagnostic tools and the personalization of cancer treatment, the incidence of cancer and cancer-related death are still growing, with drug resistance, efficient cancer cell targeting, and drug availability/distributivity being keys points to address in order to develop more efficient therapies.

PNAs are highly resistant to cleavage by chemicals and enzymes due to the substrate specific nature of enzymes and therefore are not degraded inside the cells. PNAs are emerging as new tools in the market due to their applications in antisense and antigen therapies by inhibiting translation and transcription, respectively. Hence, several methods based on PNAs have been developed for designing various anti-cancer and antigen drugs, detection of mutations or modulation of PCR reactions. PNAs present some disadvantages, notably related to the cellular uptake of PNA, which led to modifications in PNA backbone or to the covalent coupling with cell penetrating peptides to improve its delivery inside the cells. However, there remains a need to further increase the binding affinity of PNAs for nucleic acid. In addition, the lack of charged phosphate groups also contributes to the hydrophobic nature of PNAs, which leads to inferior water solubility. Because of this, conventional PNAs cannot efficiently cross cellular membranes. There is still a need for further improved PNA agents.

### SUMMARY OF THE INVENTION

The present invention is based on the seminal discovery that incorporation of cyclic structural moieties such as tetrahydrofuran, pyrrolidine, N-methyl pyrrolidine, or pyrrolidinium into C2-C3 position of a PNA monomer can lead to PNAs with improved water solubility and stronger binding affinity with nucleic acids.

In one embodiment, the present invention provides a PNA monomer derivative with a structure of formula (I) or an optically pure stereoisomer, pharmaceutically acceptable salt, or solvate thereof.

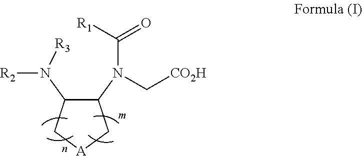

In certain aspects, n is an integer selected from 0 to 2, m is an integer selected from 0 to 2.

In various aspects, A is selected from the group consisting of —O—, —S—, —NR₄—, and

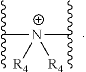

R₁ is selected from the group consisting of

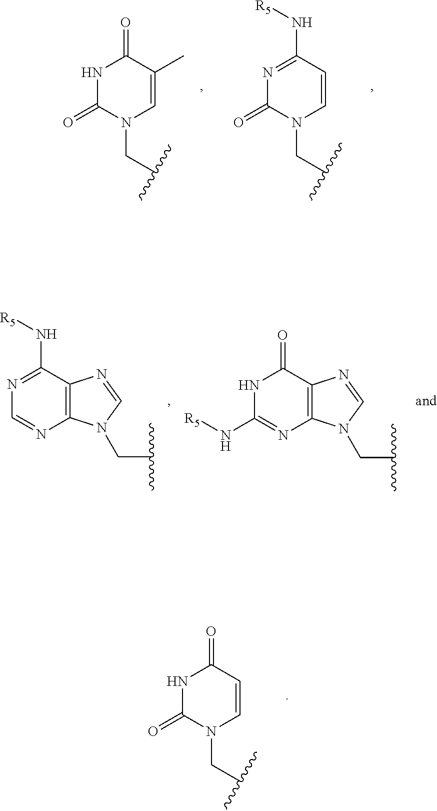

Each R₂ and R₅ is independently H or a protective group, the protective group is selected from the group consisting of tert-butyloxycarbonyl (Boc), fluorenylmethoxycarbonyl (Fmoc), benzothiazole-2-sulfonyl (Bts), allyloxycarbonyl (Alloc), carboxybenzyl (Cbz), p-nitrophenyl,1-adamantyl formate, allyl formate, triphenylmethyl, benzyl, acetyl, trifluoroacetyl, and p-toluenesulfonyl.

In one aspect, R₃ is H, methyl, ethyl, or propyl. Each R₄ is independently H, methyl, ethyl, or propyl.

In some aspects, the PNA monomer derivative in the present disclosure includes, but not limited to,

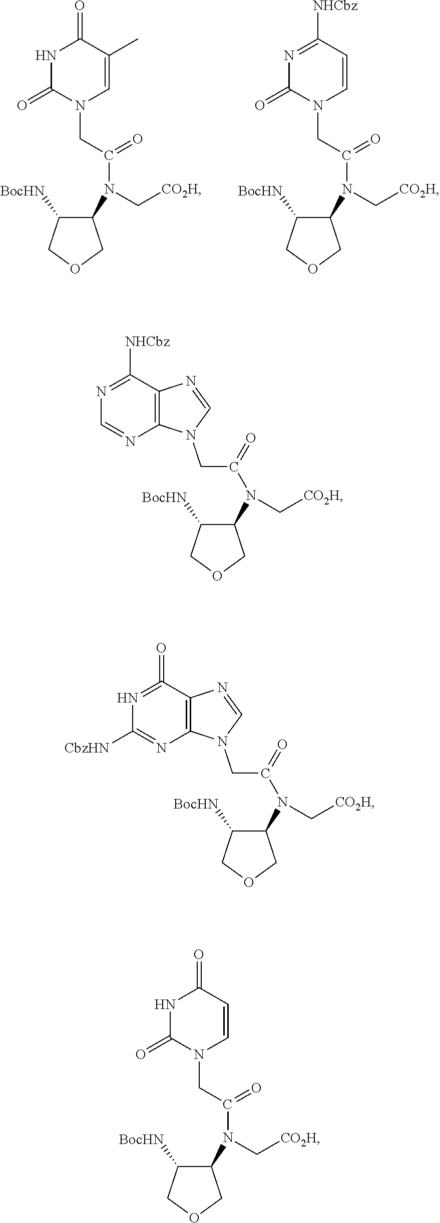

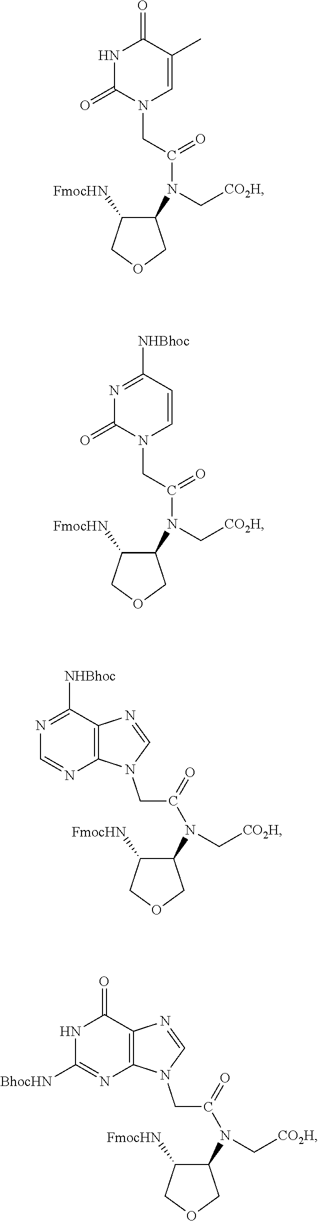

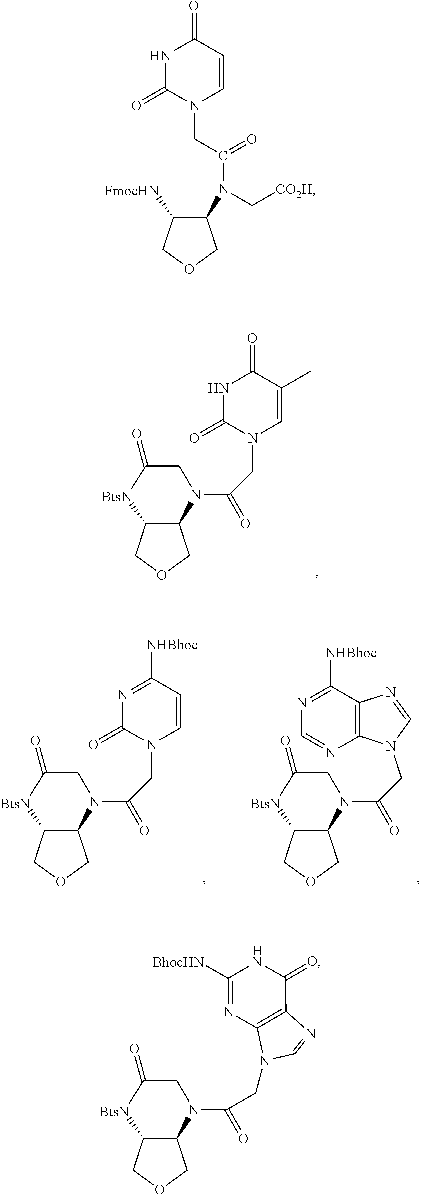

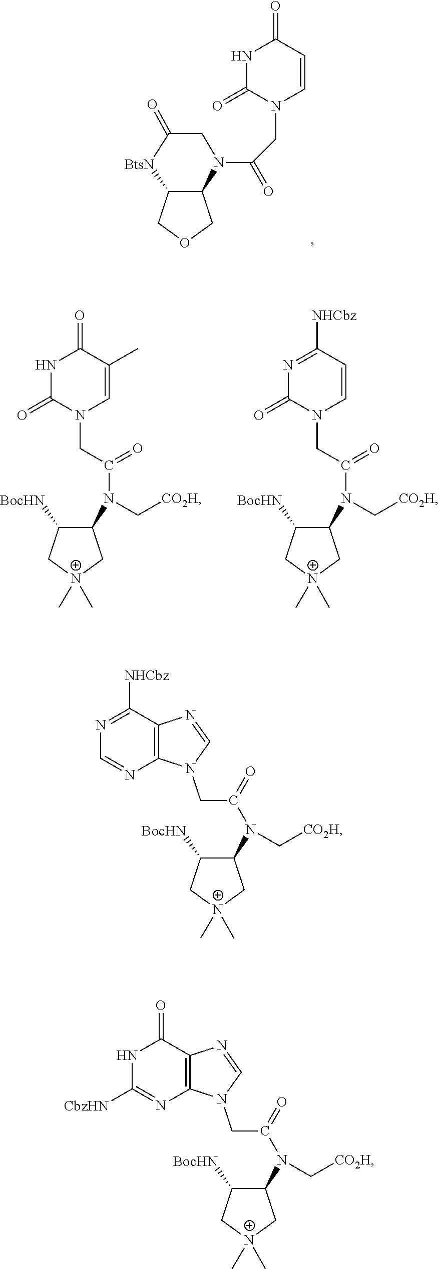

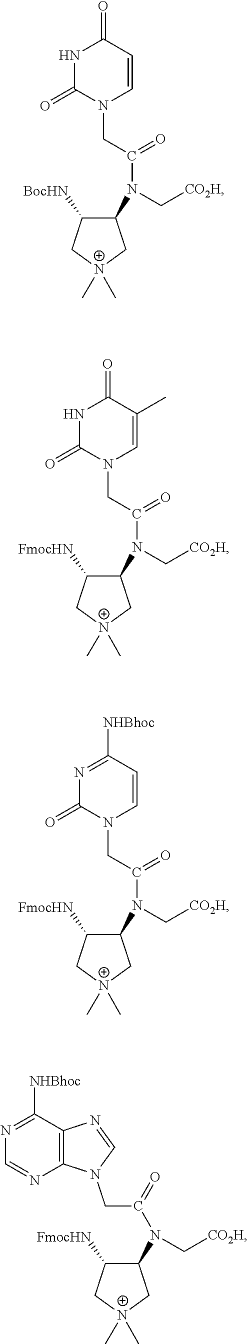

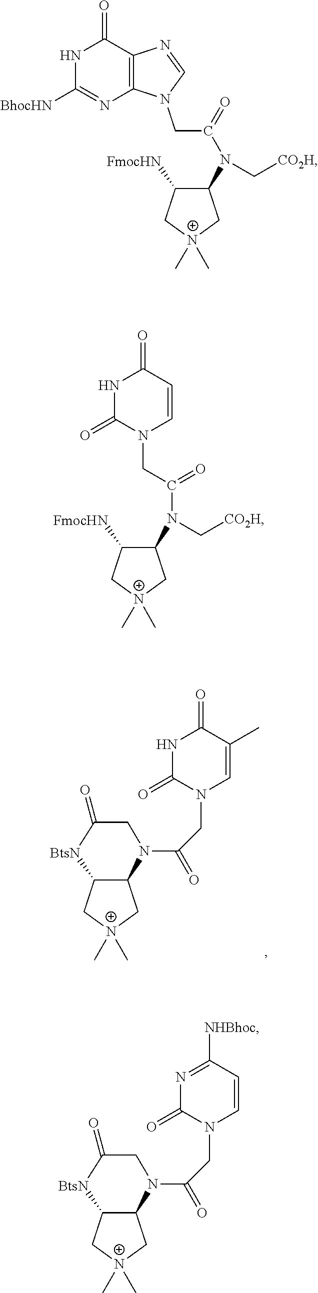

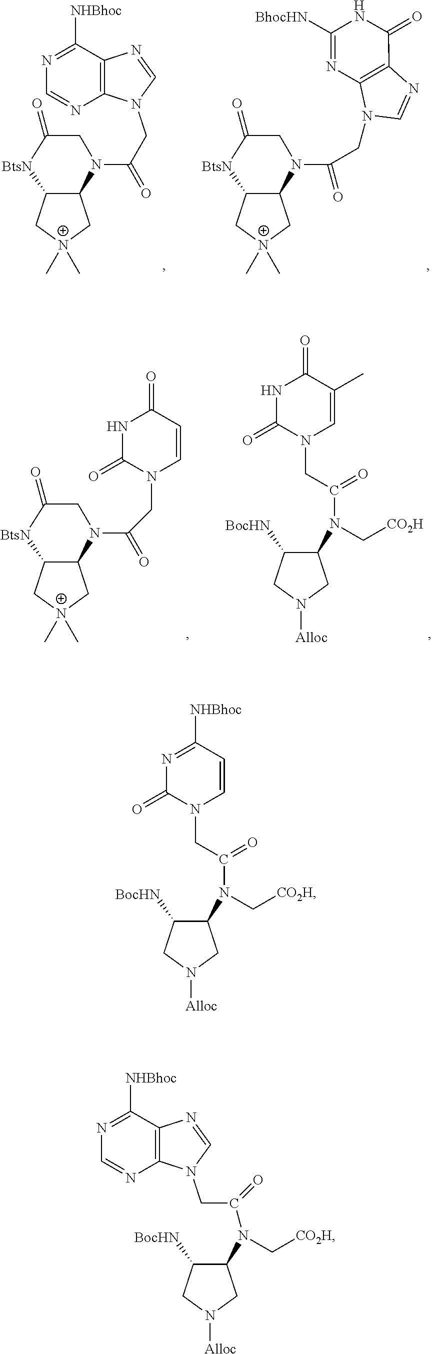

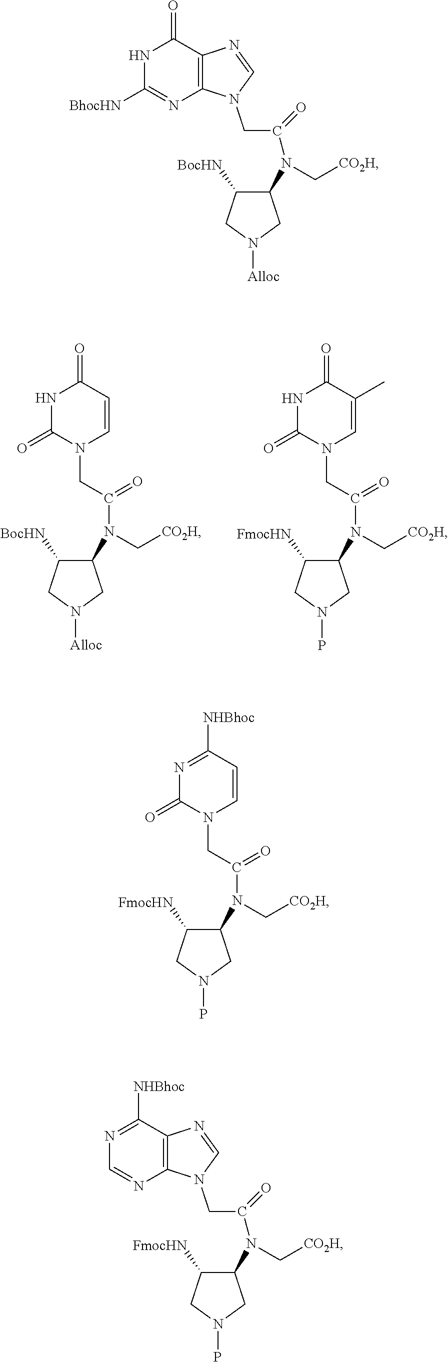

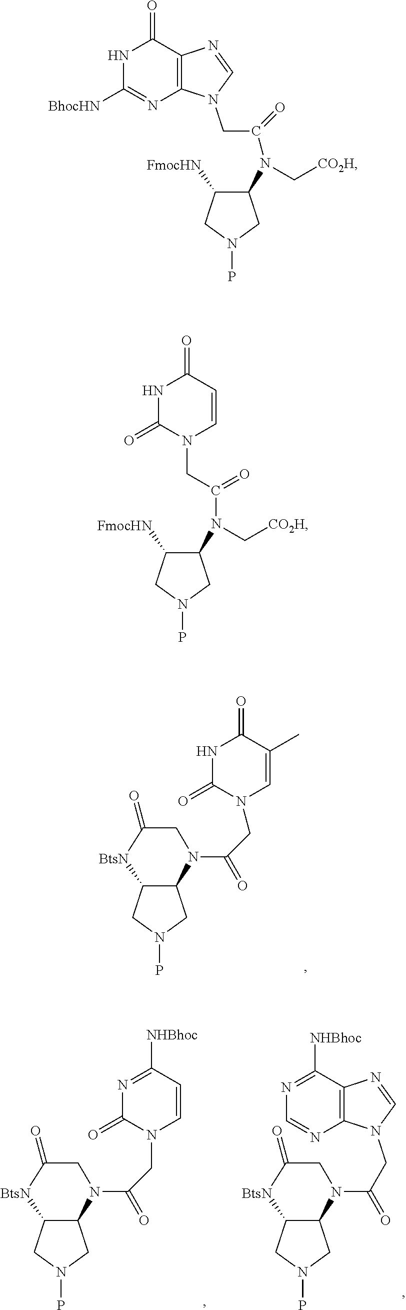

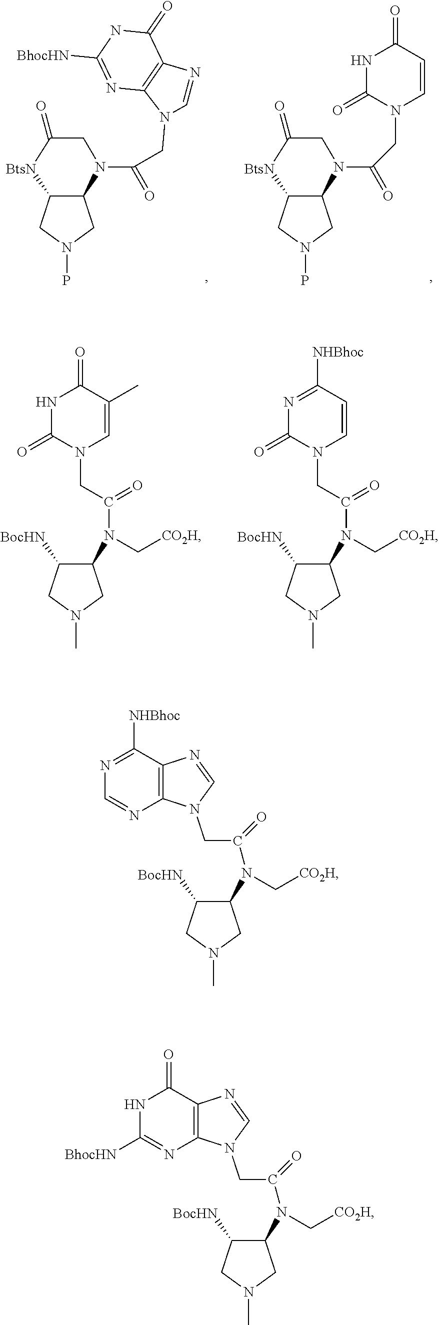

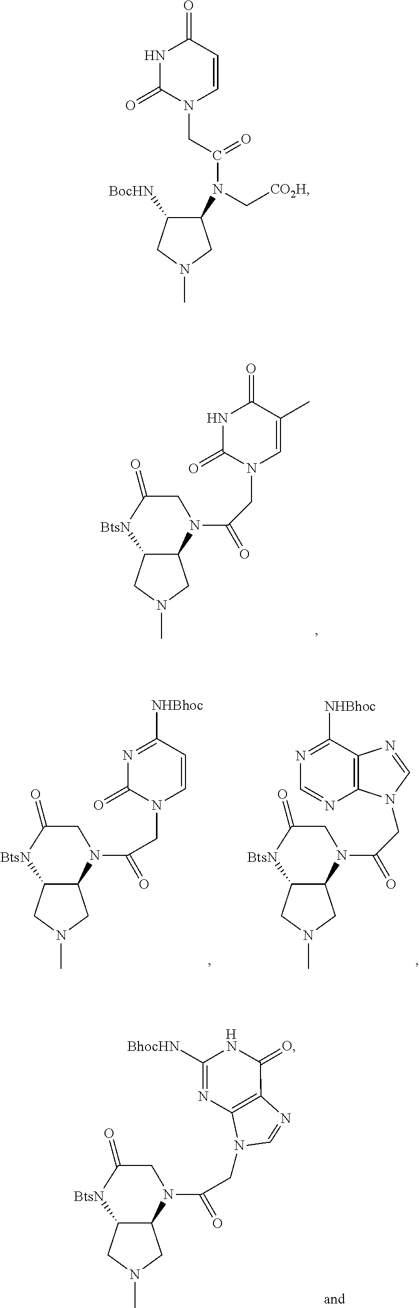

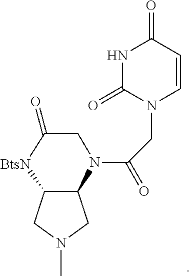

In some embodiments, P can be Boc or Alloc.

In another embodiment, the invention provides a peptide nucleic acid with a structure according to formula (II) or an optically pure stereoisomer, pharmaceutically acceptable salt, or solvate thereof.

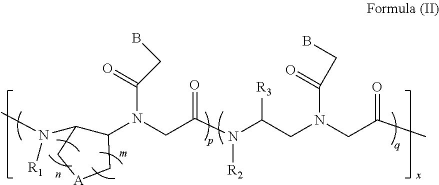

In certain aspects, each n and m is an integer independently selected from 0 to 2, each p, q, and x is an integer independently selected from 0 to 20.

In various aspects, A is selected from the group consisting of —O—, —S—, —NR₄—, and

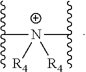

B is selected from the group consisting of

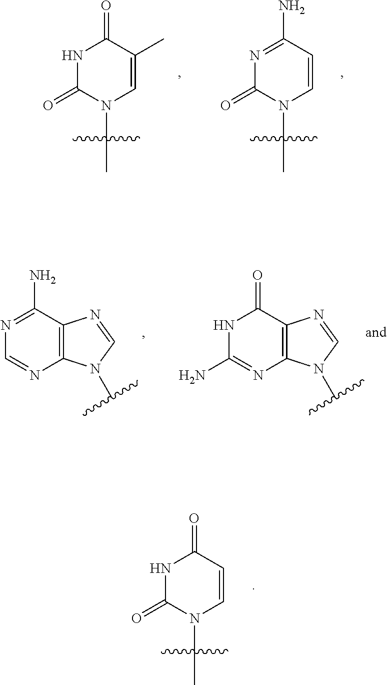

In one aspect, each R₁, R₂ and R₄ is independently H, methyl, ethyl, or propyl. R₃ is H, methyl, ethyl, propyl, F, Br, Cl, CF₃, NO₂, OH, OCH₃, CN, amino group unsubstituted or substituted with methyl, ethyl, or propyl, —CH₂(OCH₂CH₂)_{y}OMe, and

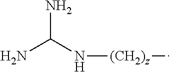

In some aspects, y is an integer selected from 0 to 5. z is an integer selected from 1 to 5.

In another embodiment, the invention provides a method of improving solubility and/or nucleic acid affinity of a peptide nucleic acid (PNA) including incorporating one or more cyclic structural moieties into a PNA monomer. In some aspects, the one or more cyclic structural moieties include tetrahydrofuran, pyrrolidinium, pyrrolidine, or N-methyl pyrrolidine moieties. In other aspects, the tetrahydrofuran, pyrrolidinium, pyrrolidine, or N-methyl pyrrolidine moieties are incorporated into C2-C3 position.

In one embodiment, the invention provides a pharmaceutical composition including the PNA with a structure according to formula (II) and a pharmaceutically acceptable carrier.

In an additional embodiment, the invention provides a method of reducing expression of a target gene in a cell including contacting a cell in which the target is expressed with a PNA agent with a structure according to formula (II).

In a further embodiment, the invention provides a method for identifying and/or characterizing PNA agents for target inhibition including: contacting a system in which a target is expressed with a PNA agent with a structure according to formula (II); determining a level or activity of the target in the system when the PNA agent is present as compared with a target reference level or activity observed under otherwise comparable conditions when it is absent; and classifying the PNA agent as a target inhibitor if the level or activity of the target is significantly reduced when the PNA agent is present as compared with the target reference level or activity.

In one embodiment, the present invention provides a method for treating cancer in a subject including administering a PNA agent with a structure of formula (II) to the subject. In certain aspects, the treatment method further includes administering an anti-cancer treatment. The PNA agent can be administered prior to, simultaneously with or following the administration of the anti-cancer treatment. In some aspects, the PNA agent can be administered orally, parenterally, intradermally, transdermally, or by inhalation.

### BRIEF DESCRIPTION OF THE DRAWINGS

FIG. 1 shows the tetrahydrofuran dihedral clamp 3D structure highlighted against a conventional PNA oligomer.

FIG. 2 shows the N-Mez pyrrolidinium dihedral clamp 3D structure highlighted against a conventional PNA oligomer.

### DETAILED DESCRIPTION OF THE INVENTION

The present invention is based on the seminal discovery that incorporation of cyclic structural moieties such as tetrahydrofuran or pyrrolidinium into C2-C3 position of a PNA monomer can lead to PNAs with improved water solubility and stronger binding affinity with nucleic acids. Moreover, the cationic and configurationally helix-guiding properties of these PNA analogues described here can create sufficient efficacy by merely placing them intermittently within a standard aegPNA oligomer. Likewise, an oligomer comprised of varying proportions of any combination of these PNA analogues and standard aegPNA would also confer sufficient efficacy of these properties. Homopolymers of any of these analogues may also confer sufficient efficacy of these properties.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a more soluble derivation of a configurationally restricted peptide nucleic acid monomer for better helical configuration. It has been shown that including one or more (S,S)-trans-cyclopentane diamine units into aminoethylglycine peptide nucleic acids (aegPNAs) significantly augments binding affinity and sequence specificity to complementary DNA. See Nielsen et al. (1991) Science 254:1497 and Bustin et al. (2002) Trends Mol. Med. 8:269. With much of the human genome sequence known in addition to bacterial and viral sequences, improved binding of PNA oligomers lends itself well towards development of improved therapeutics and diagnostic reagents.

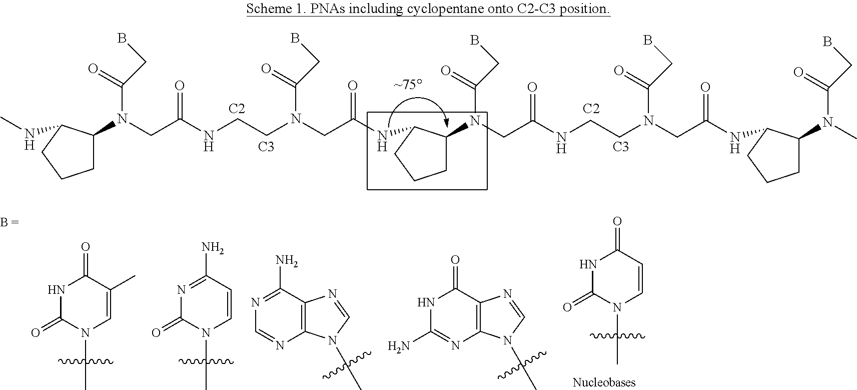

Currently, there are relatively few configurational modifications that have successfully generated PNA oligomers with improved binding affinity over the original, unmodified backbone. The most successful investigations into constraining the PNA backbone include incorporating a cyclic ring into the C2-C3 bond of the PNA frame, shown in Scheme 1 above. Nielsen and co-workers initially examined this strategy by incorporating a cyclohexane ring at this position where the diaminocyclohexane (available as a single enantiomer in either the (R,R) or (S,S) form), however this did not yield satisfactory improvements. See Lagriffoule et al. (1997) Chem. Eur. J. 3:912. Appella further investigated the geometry of what is required for effective positioning to maintain the dihedral angle of the B-DNA helical binding configuration, leading to the finding that a cyclopentane ring constraint of S,S configuration approximates this most closely. See Pokorski et al. (2004) J. Am. Chem. Soc. 126:15067-73 and Myers et al. (2003) Org. Lett. 5(15):2695-98.

From NMR data of PNA-DNA and PNA-RNA duplexes, the preferential dihedral angles of the C2-C3 bond axis are 130-165° and 60-80°. Molecular mechanics studies show available dihedrals for trans configured bonds at ˜80° and 160°, very near those measured by NMR experiments for the substituents located across the C2-C3 bond axis. Incorporation of a (1S, 2S) trans diaminopentane into PNA oligomers to replace the ethylenediamine link have shown higher melting temperatures with complementary DNA oligomers consistent with stronger binding.

One of the main concerns of PNA oligomers are their solubility limitations under physiological conditions. These limitations can be overcome by addition of cationic moieties such as lysines and/or arginines to allow their aqueous dissolution. However, improving solubility characteristics of the PNA oligomer itself should offer a better solution to this issue. Moreover, such incorporated configurationally stabilizing cyclopentane moieties also add insoluble aliphatic properties to the PNA oligomer outright by adding a —CH₂—CH₂—CH₂— to each monomer. The present disclosure submits modifications to this cyclopentane configurational clamp incorporating chemical features that allow it improved solubility with the potential to allow further facile modification. Solubility is also aided by the cationic charge of the pyrrolidinium —CH₂—⁺NR₂—CH₂—, N-methyl pyrrolidine —CH₂—⁺NHR—CH₂—, and pyrrolidine —CH₂—⁺NH₂—CH₂— in addition to affinity to the anionic DNA polymer, and ability to aid transfer and delivery across phospholipid bilayers of cell and nuclear membranes.

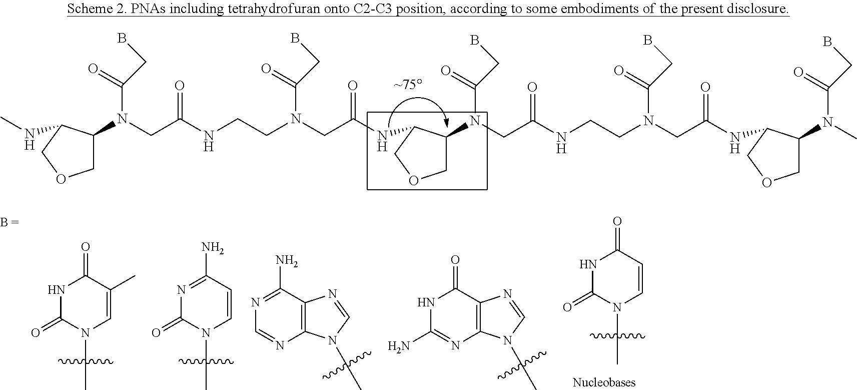

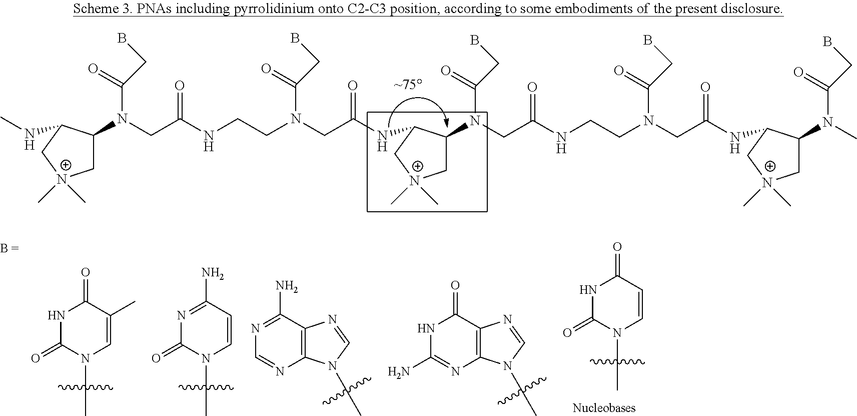

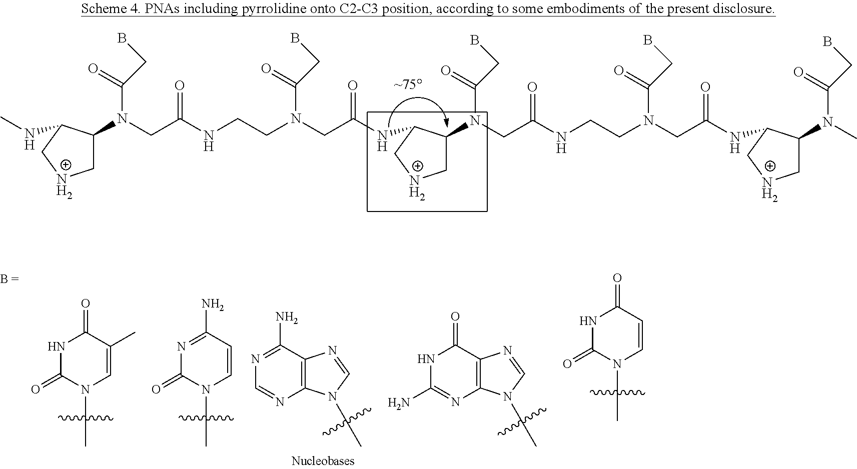

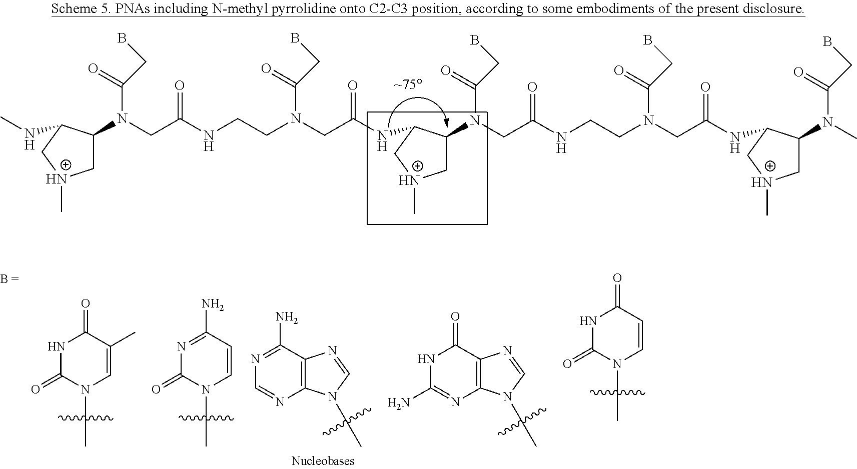

Replacing the apical 4-methylene of the cyclopentane with an ether oxygen as —CH₂—O—CH₂— or quaternary alkyl ammonium moiety as —CH₂—N⁺(CH₃)₂—CH₂— or the amine as —CH₂—N⁺H₂—CH₂— or the N-methyl amine as —CH₂—N⁺HMe-CH₂— offers improved solubility as it lies at the accessible surface of the PNA oligomeric structure. The former adds as a tetrahydrofuran incorporated as (3R, 4R) 3,4-trans-tetrahydrofurandiamine (Scheme 2) and the latter adds as a pyrrolidinium incorporated as (3R, 4R) 3,4-trans-diamino 1,1-dimethyl pyrrolidinium (Scheme 3), the next adds as a pyrrolidine incorporated (3R, 4R) 3,4-trans-pyrrolidine (Scheme 4), and the next adds as the N-methyl pyrrolidine incorporated (3R, 4R) 3,4-trans-N-methyl pyrrolidine (Scheme 5), which are known completely miscible in water, unlike cyclopentane which as an alkane is immiscible with water. Moreover, these modifications maintain similar tetrahedral bond angle configurations of the thoroughly vetted cyclopentane structure.

Other means to impart solubility and structural constraint to confer structure to a geometry closely approximating its binding configuration include g-(S) substituted PNA. See Sahu et al. (2009) J. Org. Chem. 74:1509-16 and Sahu et al. (2011) J. Org. Chem. 76:5614-27. These incorporate γ-lysine-amine, arginine-guanidinium, polyethylene glycol (PEG) sidechains that are large and function by steric limitation of its dihedral rotation to favor a configuration to better approximate binding complementary DNA target in the natural B-DNA helical configuration. Limitations to this approach include a larger moiety appended at one end offering a more configurationally labile occupation of space. Limitations also include more steric obstructions to binding target complementary DNA strand, as well as difficulties for incorporation into standard PNA oligomer synthesis.

Synthesis of the Boc, Fmoc and Bts protected monomers for the tetrahydrofuran-based, pyrrolidinium-based, pyrrolidine-based or N-methyl pyrrolidine-based PNAs make them easily accessible for their incorporation into PNA oligomers by standard Boc-based, Fmoc-based or Bts-based solid phase peptide synthesis. Synthesis of the tetrahydrofuran-based derivative can be accessed through commercially available t-butyl N-[(3R, 4R)-4-aminooxolan-3-yl] carbamate (compound 1). Synthesis of the pyrrolidinium-based derivative can be accessed through commercially available 1-methyl, (3R, 4R)-3,4 pyrrolinediol. The pyrrolidine-based derivative can be accessed through commercially available (3S, 4S)-tert-butyl 3,4-diaminopyrroldidine-1-carboxylate to create the Boc-protected pyrrolidine derivative and (3S, 4S)-benzyl 3,4-diaminopyrrolidine-1-carboxylate to create the Alloc-protected pyrrolidine-based derivative.

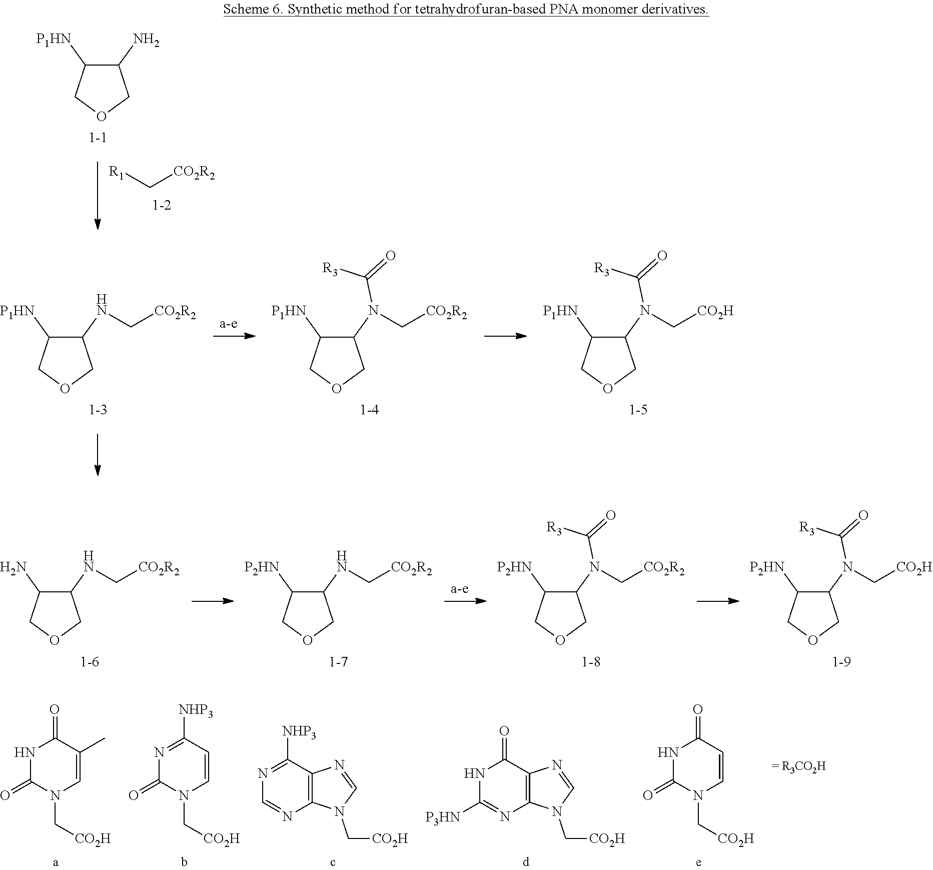

In some embodiments, tetrahydrofuran-based PNA monomer derivatives, with different protective groups such as P1 (compound 1-4) and P2 (compound 1-8), can be synthesized according to scheme 6. The synthesis can start with compound 1-1, with 3-amino protected by P1, while 4-amino can be reacted with compound 1-2 to obtain intermediate 1-3, which can be further reacted with compounds a-e to introduce nucleobases such as thymine (a), cytosine (b), adenine (c), guanine (d) and uracil (e). R₁ is a halogen. R₂ is an alkyl or allyl. The alkyl ester intermediate 1-4 can be hydrolyzed or the allyl ester deprotected in the final step to generate P1 protected PNA monomer derivatives 1-5.

In alternative, if a different protective group, P2, is preferred, P1-protected intermediate 1-3 can be deprotected to generate free amine intermediate 1-6, which is further protected with P2 to obtain intermediate 1-7. Similar reactions can be followed to introduce nucleobases moieties to generate intermediate compound 1-8, which is hydrolyzed to obtain the P2-protected PNA monomer derivatives 1-9.

In some embodiments, for both derivatives 1-5 and 1-9, when P1 and/or P2 is certain protective group, a further lactamization can occur between —CO2H and —P1NH (or —P2NH). In some embodiments, such lactamization reaction can occur when P1 and/or P2 is Bts.

Each P1, P2, and P3 can be a protective group for the amino group independently selected from the group consisting of tert-butyloxycarbonyl (Boc), fluorenylmethoxycarbonyl (Fmoc), Benzothiazole-2-sulfonyl (Bts), allyloxycarbonyl (Alloc), carboxybenzyl (Cbz), benzhydryloxycarbonyl (Bhoc), p-nitrophenyl, 1-adamantyl formate, allyl formate, triphenylmethyl, benzyl, acetyl, trifluoroacetyl, and p-toluenesulfonyl.

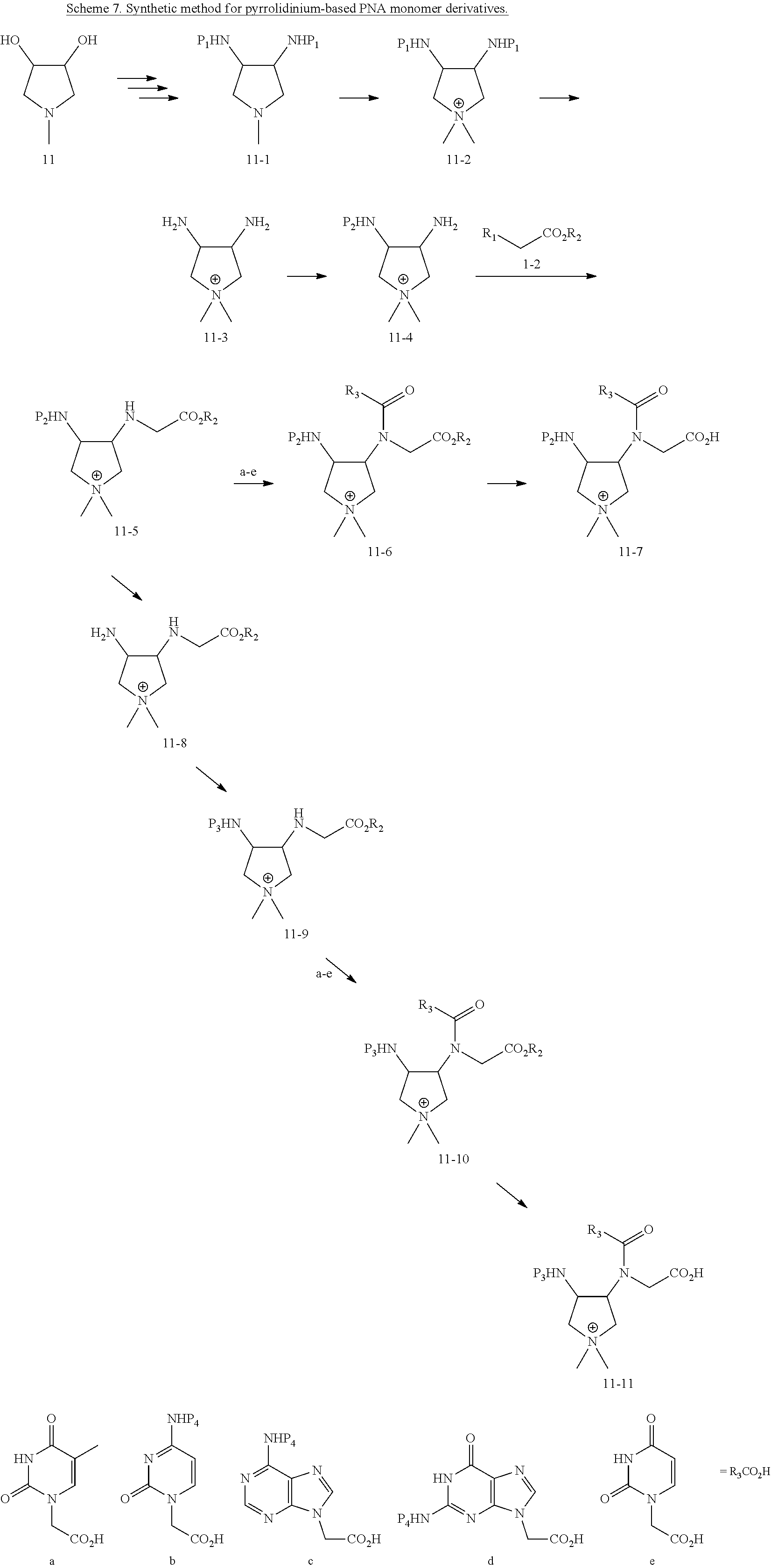

In some embodiments, pyrrolidinium-based PNA monomer derivatives, with different protective groups such as P2 (compound 11-7) and P3 (compound 11-11), can be synthesized according to scheme 7. The synthesis can start with commercially-available compound 11, 1-methyl, (3R, 4R)-3,4 pyrrolinediol, which can be converted to di-P1-protected tertiary amine intermediate 11-1. The tertiary amine intermediate 11-1 can be converted to a quaternary ammonium intermediate 11-2. After deprotection to get the diamino ammonium intermediate 11-3, one of the amines can be protected via P2 to get intermediate 11-4. Intermediate 11-4 can be reacted with compound 1-2 to obtain ester intermediate 11-5, which can be further reacted with compounds a-e to introduce nucleobases such as thymine (a), cytosine (b), adenine (c), guanine (d) and uracil (e). R₁ is a halogen. R₂ is an alkyl. The ester intermediate 11-6 can be selectively hydrolyzed in the final step to generate P2-protected PNA monomer derivatives 11-7.

In alternative, if a different protective group, P3, is preferred, P2-protected intermediate 11-5 can be deprotected to generate free amine intermediate 11-8, which is further protected with P3 to obtain intermediate 11-9. Similar reactions can be followed to introduce nucleobase moieties to generate intermediate compound 11-10, which is selectively hydrolyzed to obtain the P3-protected PNA monomer derivatives 11-11. In some embodiments, for both derivatives 11-7 and 11-11, when P2 and/or P3 is certain protective group, a further lactamization can occur between —CO2H and —P2NH (or —P3NH). In some embodiments, such a lactamization reaction can occur when P2 and/or P3 is Bts.

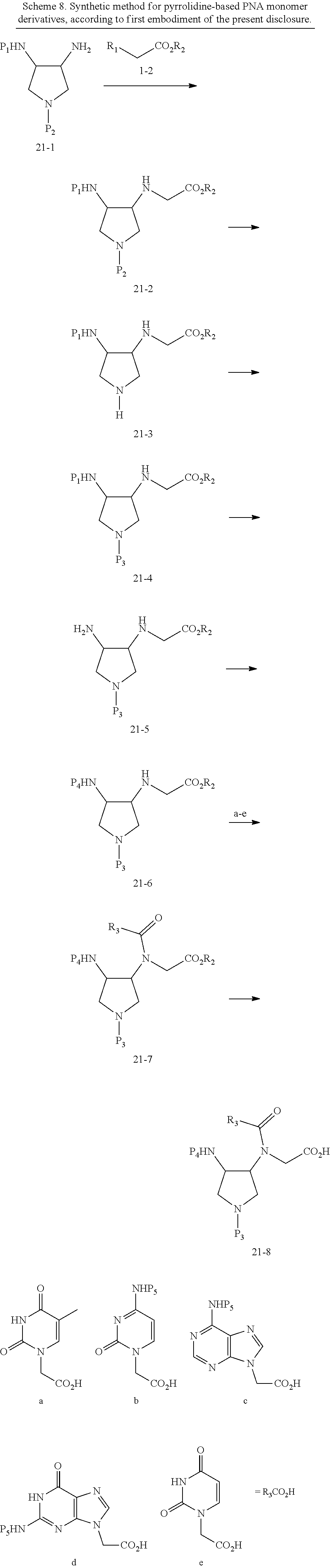

In a first embodiment of the present disclosure, pyrrolidine-based PNA monomer derivatives can be synthesized according to scheme 8. The synthesis can start with compound 21-1, with 3-amino protected by P1 and the pyrrolidine nitrogen protected by P2. The 4-amino can be reacted with compound 1-2 to obtain intermediate 21-2, which can be deprotected to form intermediate 21-3 with deprotected pyrrolidine nitrogen. Intermediate 21-3 can undergo further reaction to protect the pyrrolidine nitrogen with P3 to form intermediate 21-4, which can undergo deprotection chemistry to on the 3-amino to form intermediate 21-5. Intermediate 21-5 can undergo protection chemistry to protect 3-amino with P4, which can further react with compounds a-e to introduce nucleobases such as thymine (a), cytosine (b), adenine (c), guanine (d) and uracil (e). R₁ is a halogen. R₂ is an alkyl. The ester intermediate 21-7 can be selectively hydrolyzed in the final step to generate PNA monomer derivatives 21-8. In some embodiments, P3 is Alloc protective group. In some embodiments, when P4 is certain protective group, a further lactamizaiton can occur between —CO2H and —P4NH. In some embodiments, such a lactamization reaction can occur when P4 is Bts.

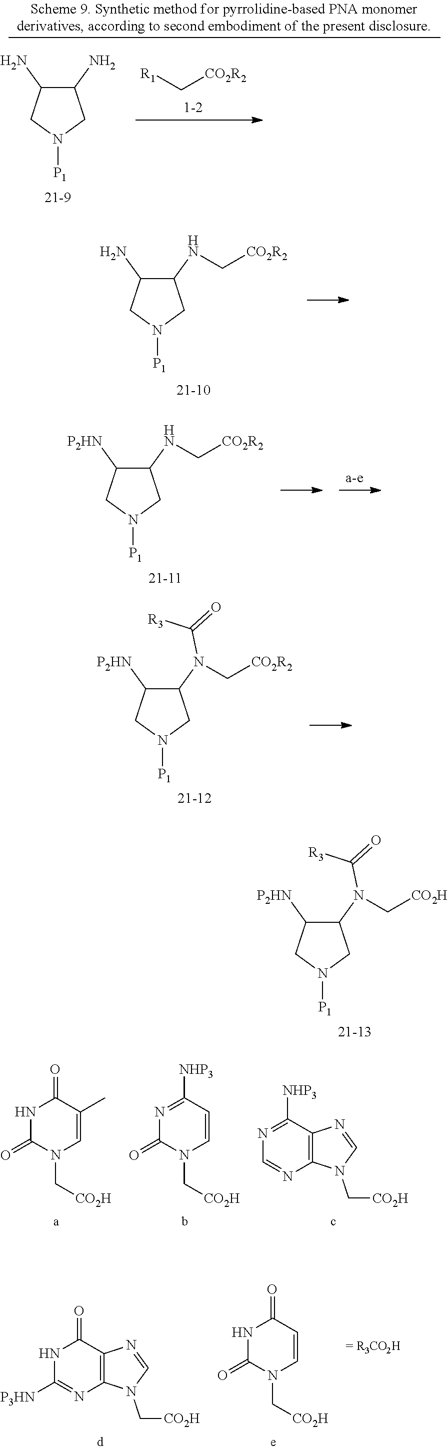

In a second embodiment of the present disclosure, pyrrolidine-based PNA monomer derivatives can be synthesized according to scheme 9. The synthesis can start with compound 21-9, with pyrrolidine nitrogen protected by P1, which can react with compound 1-2 to obtain intermediate 21-10, which can undergo protection chemistry to form intermediate 21-11 with the 4-amino position protected by P2. Intermediate 21-11 can further react with compounds a-e to introduce nucleobases such as thymine (a), cytosine (b), adenine (c), guanine (d) and uracil (e). R₁ is a halogen. R₂ is an alkyl. The ester intermediate 21-12 can be selectively hydrolyzed in the final step to generate PNA monomer derivatives 21-13. In some embodiments, P1 is Boc protective group. In some embodiments, when P2 is certain protective group, a further lactamization can occur between —CO2H and —P4NH. In some embodiments, such a lactamization reaction can occur when P2 is Bts.

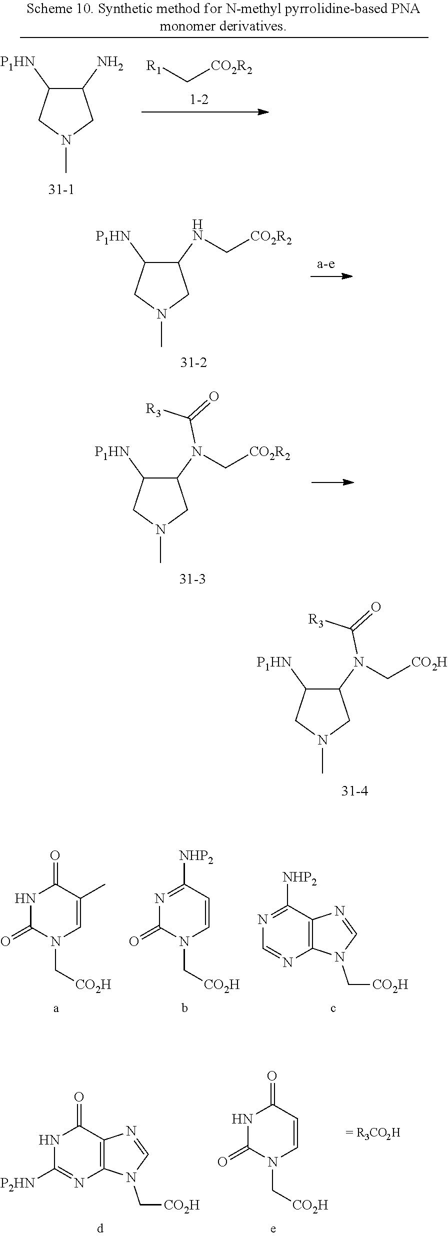

In some embodiments, N-methyl pyrrolidine-based PNA monomer derivatives, with different protective groups such as P1 can be synthesized according to scheme 10. The synthesis can start with compound 31-1, with 3-amino protected by P1, while 4-amino can be reacted with compound 1-2 to obtain intermediate 31-2, which can be further reacted with compounds a-e to introduce nucleobases such as thymine (a), cytosine (b), adenine (c), guanine (d) and uracil (e). R₁ is a halogen. R₂ is an alkyl. The ester intermediate 31-3 can be selectively hydrolyzed in the final step to generate P1 protected PNA monomer derivatives 31-4. In some embodiments, when P1 is certain protective group, a further lactamizaiton can occur between —CO2H and —P1NH. In some embodiments, such a lactamization reaction can occur when P1 is Bts.

Each P1, P2, P3, and P4 can be a protective group for the amino group independently selected from the group consisting of tert-butyloxycarbonyl (Boc), fluorenylmethoxycarbonyl (Fmoc), Benzothiazole-2-sulfonyl (Bts), allyloxycarbonyl (Alloc), carboxybenzyl (Cbz), benzhydryloxycarbonyl (Bhoc), p-nitrophenyl, 1-adamantyl formate, allyl formate, triphenylmethyl, benzyl, acetyl, trifluoroacetyl, and p-toluenesulfonyl.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched-chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C amido” as used herein, alone or in combination, refers to a C(═O) NR₂ group with R as defined herein. The term “N amido” as used herein, alone or in combination, refers to a RC(═O)NH group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkane carboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C6H4=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O carbamyl” as used herein, alone or in combination, refers to a OC(O)NRR′ group with R and R′ as defined herein.

The term “N carbamyl” as used herein, alone or in combination, refers to a ROC(O)NR′ group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O carboxy” group refers to a RC(O)O— group, where R is as defined herein. A “C carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl. 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene.

(—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either: 1) monocyclic heteroaryl comprising five or six ring members, of which between one and four said members may be heteroatoms selected from the group consisting of O, S, and N; or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N sulfonamido” refers to a RS(═O)₂NR′ group with R and R′ as defined herein.

The term “S sulfonamido” refers to a S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N thiocarbamyl” refers to an ROC(S)NR′— group, with R and R′ as defined herein.

The term “O thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rₙ where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “optically pure stereoisomer” refers to stereosiomeric, such as enantiomeric or diastereomeric excess or the absolute difference between the mole fraction of each enantiomer or diastereomer.

The present invention is based on the seminal discovery that incorporation of tetrahydrofuran or pyrrolidinium into C2-C3 position of a PNA monomer can lead to PNAs with improved water solubility and stronger binding affinity with nucleic acids.

In one embodiment, the present invention provides a PNA monomer derivative with a structure of formula (I) or an optically pure stereoisomer, pharmaceutically acceptable salt, or solvate thereof.

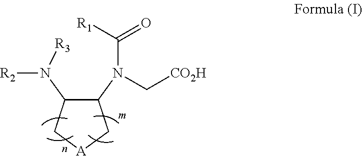

In certain aspects, n is an integer selected from 0 to 2, m is an integer selected from 0 to 2.

In various aspects, A is selected from the group consisting of —O—, —S—, —NR₄—, and

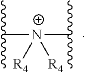

R₁ is selected from the group consisting of

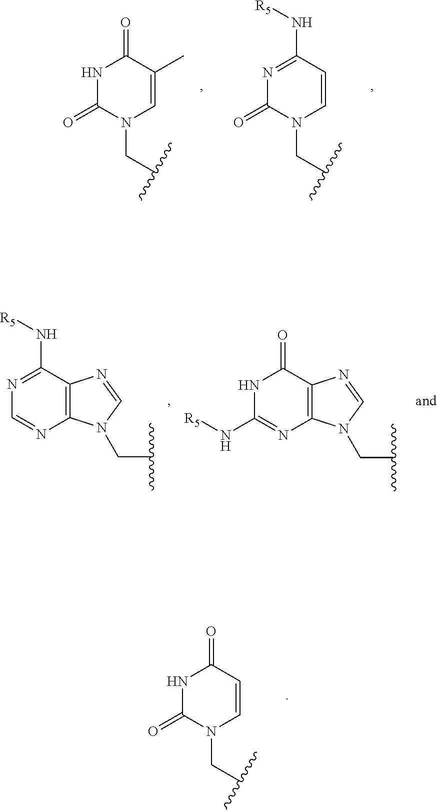

Each R₂ and R₅ is independently H or a protective group, the protective group is selected from the group consisting of tert-butyloxycarbonyl (Boc), fluorenylmethoxycarbonyl (Fmoc), benzothiazole-2-sulfonyl (Bts), allyloxycarbonyl (Alloc), carboxybenzyl (Cbz), p-nitrophenyl, 1-adamantyl formate, allyl formate, triphenylmethyl, benzyl, acetyl, trifluoroacetyl, and p-toluenesulfonyl.

In some aspects, protective group for the amino group used in the present disclosure includes, but is not limited to, tert-butyloxycarbonyl (Boc), fluorenylmethoxycarbonyl (Fmoc), carboxybenzyl (Cbz), benzhydryloxycarbonyl (Bhoc), p-nitrophenyl, 1-adamantyl formate, allyl formate, triphenylmethyl, benzyl, acetyl, trifluoroacetyl, and p-toluenesulfonyl.

tert-butyloxycarbonyl (Boc) refers to

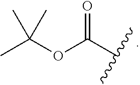 [SMILES: *C(=O)OC(C)(C)C]

Benzothiazole-2-sulfonyl (Bts) refers to

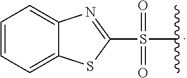 [SMILES: *S(=O)(=O)c1nc2ccccc2s1]

Fluorenylmethoxycarbonyl (Fmoc) refers to

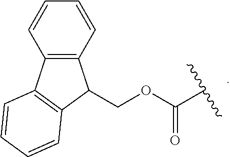 [SMILES: *C(=O)OCC1c2ccccc2-c2ccccc21]

Carboxybenzyl (Cbz) refers to

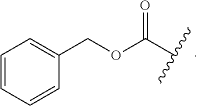 [SMILES: *C(=O)OCc1ccccc1]

Benzhydryloxycarbonyl (Bhoc) refers to

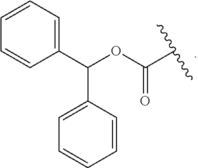 [SMILES: *C(=O)OC(c1ccccc1)c1ccccc1]

p-nitrophenyl refers to

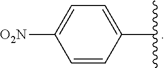 [SMILES: *c1ccc([N+](=O)[O-])cc1]

1-adamantyl formate refers to

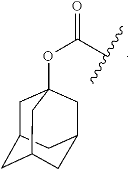 [SMILES: *C(=O)OC12CC3CC(CC(C3)C1)C2]

Allyl formate or Allyloxycarbonyl (Alloc) refers to

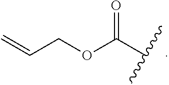 [SMILES: *C(=O)OCC=C]

Triphenylmethyl refers to

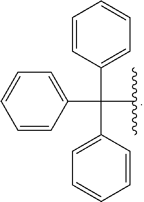 [SMILES: *C(c1ccccc1)(c1ccccc1)c1ccccc1]

Benzyl refers to

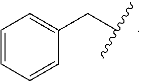 [SMILES: *Cc1ccccc1]

Acetyl refers to

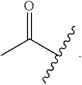 [SMILES: *C(C)=O]

Trifluoroacetyl refers to

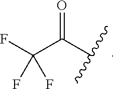 [SMILES: *C(=O)C(F)(F)F]

p-toluenesulfonyl refers to

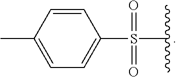 [SMILES: *S(=O)(=O)c1ccc(C)cc1]

In one aspect, R₃ is H, methyl, ethyl, or propyl. Each R₄ is independently H, methyl, ethyl, or propyl.

In certain embodiments, the present invention provides a PNA monomer derivative with a structure of formula (III) or an optically pure stereoisomer, pharmaceutically acceptable salt, or solvate thereof.

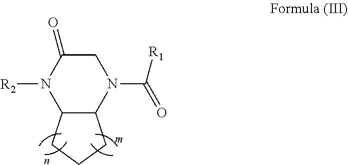

In certain aspects, n is an integer selected from 0 to 2, m is an integer selected from 0 to 2.

In various aspects, A is selected from the group consisting of —O—, —S—, —NR₃—, and

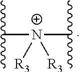

R₁ is selected from the group consisting of

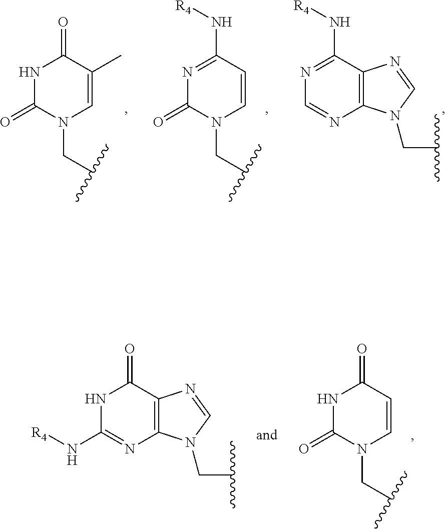

Each R₂ and R₄ is independently H or a protective group, the protective group is selected from the group consisting of tert-butyloxycarbonyl (Boc), fluorenylmethoxycarbonyl (Fmoc), benzothiazole-2-sulfonyl (Bts), allyloxycarbonyl (Alloc), carboxybenzyl (Cbz), p-nitrophenyl, 1-adamantyl formate, allyl formate, triphenylmethyl, benzyl, acetyl, trifluoroacetyl, and p-toluenesulfonyl.

Each R₃ is independently H, methyl, ethyl, or propyl. In some aspects, R₂ in Formula (III) is Bts.

In another embodiment, the invention provides a peptide nucleic acid with a structure according to formula (II) or an optically pure stereoisomer, pharmaceutically acceptable salt, or solvate thereof.

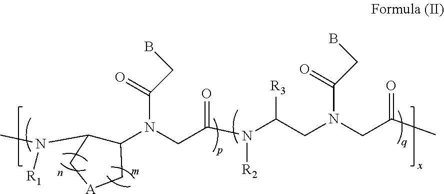

In certain aspects, each n and m is an integer independently selected from 0 to 2, each p, q, and x is an integer independently selected from 0 to 20.

In various aspects, A is selected from the group consisting of —O—, —S—, —NR₄—, and

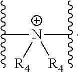

B is selected from the group consisting of

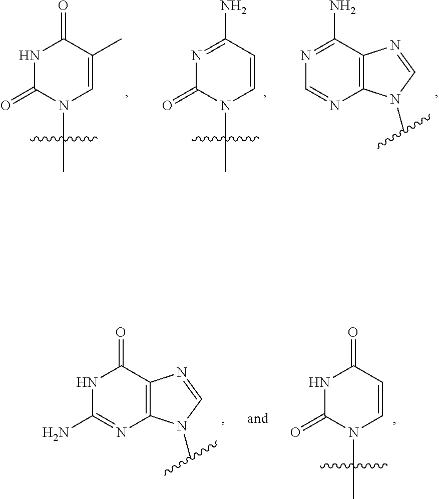

In one aspect, each R₁, R₂ and R₄ is independently H, methyl, ethyl, or propyl. R₃ is H, methyl, ethyl, propyl, F, Br, Cl, CF₃, NO₂, OH, OCH₃, CN, amino group unsubstituted or substituted with methyl, ethyl, or propyl, COOH, SO₃H, —PO(OH)₂, —OPO(OH)₂, —CH₂(OCH₂CH₂)_{y}OMe, and

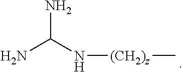

In some aspects, y is an integer selected from 0 to 5. z is an integer selected from 1 to 5.

TABLE 1
The PNA monomer derivatives in the present disclosure.

| Compound No. | Molecular Structure |
|---|---|
| Compound 5a | 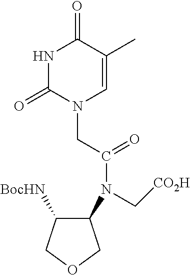 [SMILES: Cc1cn(CC(=O)N(CC(=O)O)[C@H]2COC[C@@H]2NC(=O)OC(C)(C)C)c(=O)[nH]c1=O] |
| Compound 5b | 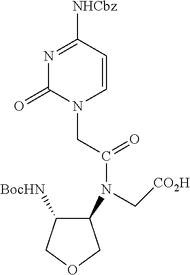 [SMILES: CC(C)(C)OC(=O)N[C@H]1COC[C@@H]1N(CC(=O)O)C(=O)Cn1ccc(NC(=O)OCc2ccccc2)nc1=O] |
| Compound 5c | 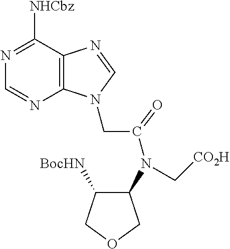 [SMILES: CC(C)(C)OC(=O)N[C@H]1COC[C@@H]1N(CC(=O)O)C(=O)Cn1cnc2c(NC(=O)OCc3ccccc3)ncnc21] |
| Compound 5d | 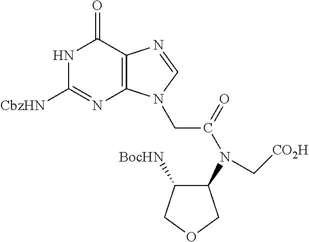 [SMILES: CC(C)(C)OC(=O)N[C@H]1COC[C@@H]1N(CC(=O)O)C(=O)Cn1cnc2c(=O)[nH]c(NC(=O)OCc3ccccc3)nc21] |
| Compound 5e | 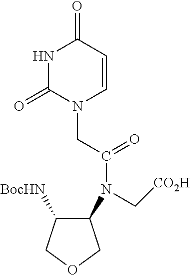 [SMILES: CC(C)(C)OC(=O)N[C@H]1COC[C@@H]1N(CC(=O)O)C(=O)Cn1ccc(=O)[nH]c1=O] |
| Compound 10a | 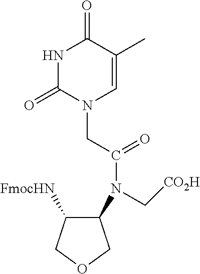 |
| Compound 10b | 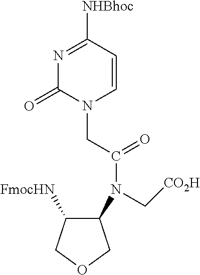 |
| Compound 10c | 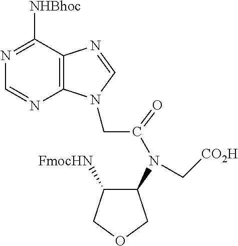 |
| Compound 10d | 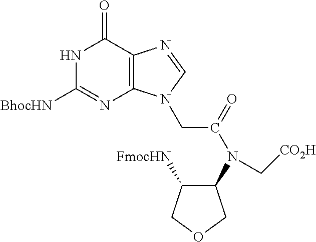 |
| Compound 10e | 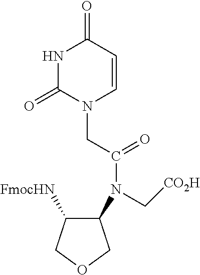 |
| Compound 14a | 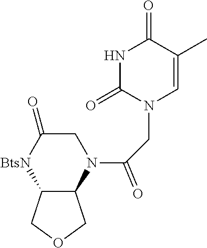 |
| Compound 14b | 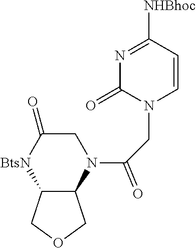 |
| Compound 14c | 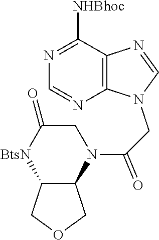 |
| Compound 14d | 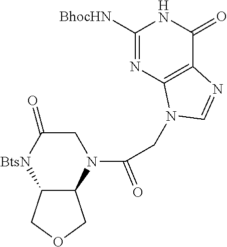 |
| Compound 14e | 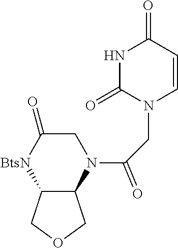 |
| Compound 20a | 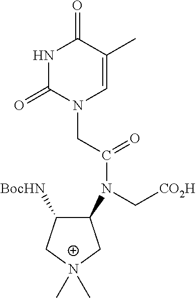 [SMILES: Cc1cn(CC(=O)N(CC(=O)O)[C@H]2C[N+](C)(C)C[C@@H]2NC(=O)OC(C)(C)C)c(=O)[nH]c1=O] |
| Compound 20b | 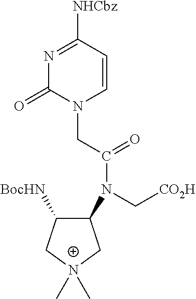 [SMILES: CC(C)(C)OC(=O)N[C@H]1C[N+](C)(C)C[C@@H]1N(CC(=O)O)C(=O)Cn1ccc(NC(=O)OCc2ccccc2)nc1=O] |
| Compound 20c | 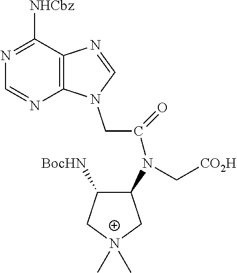 [SMILES: CC(C)(C)OC(=O)N[C@H]1C[N+](C)(C)C[C@@H]1N(CC(=O)O)C(=O)Cn1cnc2c(NC(=O)OCc3ccccc3)ncnc21] |
| Compound 20d | 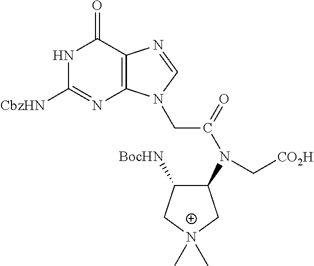 [SMILES: CC(C)(C)OC(=O)N[C@H]1C[N+](C)(C)C[C@@H]1N(CC(=O)O)C(=O)Cn1cnc2c(=O)[nH]c(NC(=O)OCc3ccccc3)nc21] |
| Compound 20e | 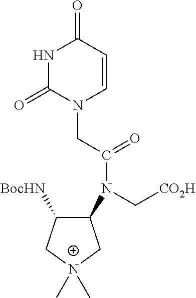 [SMILES: CC(C)(C)OC(=O)N[C@H]1C[N+](C)(C)C[C@@H]1N(CC(=O)O)C(=O)Cn1ccc(=O)[nH]c1=O] |
| Compound 22a | 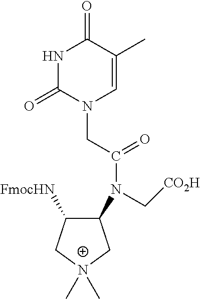 |
| Compound 22b | 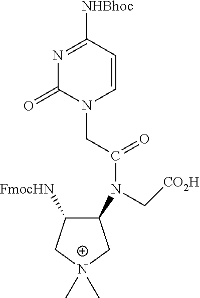 |
| Compound 22c | 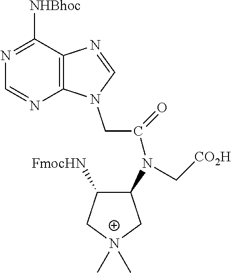 |
| Compound 22d | 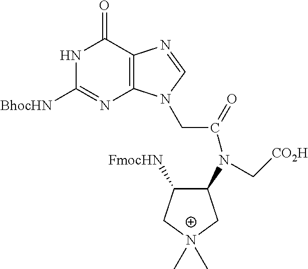 |
| Compound 22e | 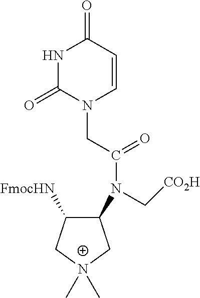 |
| Compound 32a | 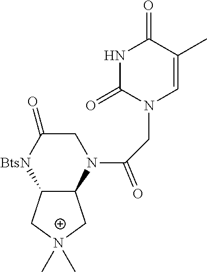 |
| Compound 32b | 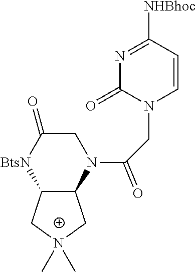 |
| Compound 32c | 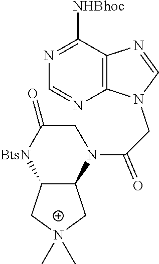 |
| Compound 32d | 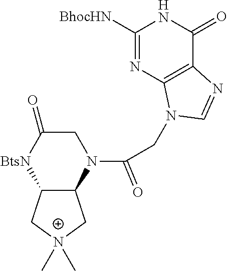 |
| Compound 32e | 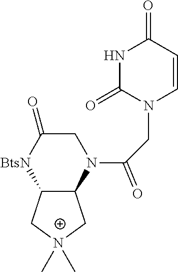 |
| Compound 49a | 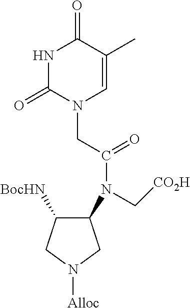 |
| Compound 49b | 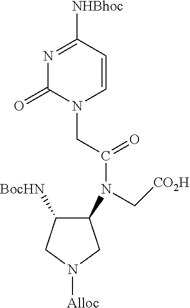 |
| Compound 49c | 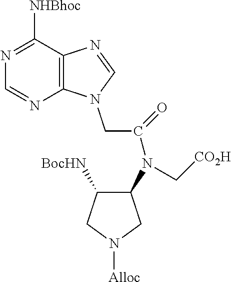 |
| Compound 49d | 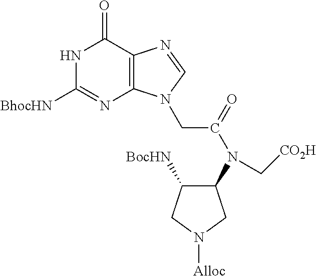 |
| Compound 49e | 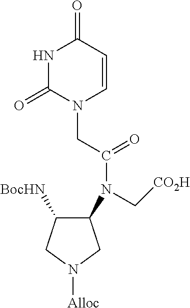 |
| Compound 50a (P = Alloc) Compound 52a (P = Boc) | 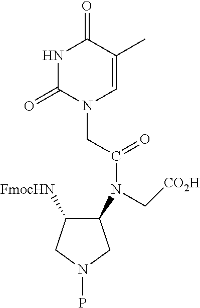 |
| Compound 50b (P = Alloc) Compound 52b (P = Boc) | 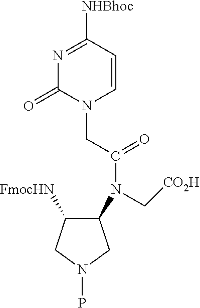 |
| Compound 50c (P = Alloc) Compound 52c (P = Boc) | 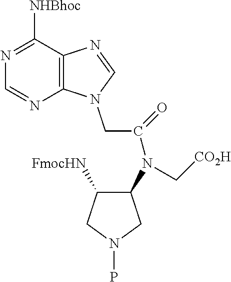 |
| Compound 50d (P = Alloc) Compound 52d (P = Boc) | 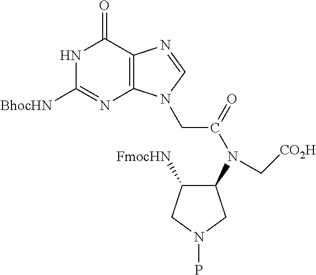 |
| Compound 50e (P = Alloc) Compound 52e (P = Boc) | 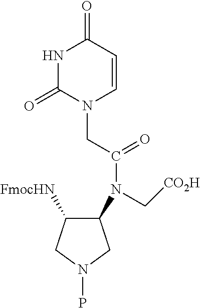 |
| Compound 54a (P = Alloc) Compound 55a (P = Boc) | 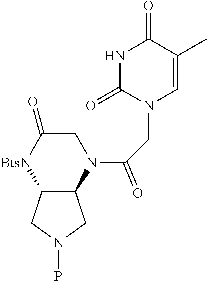 |
| Compound 54b (P = Alloc) Compound 55b (P = Boc) | 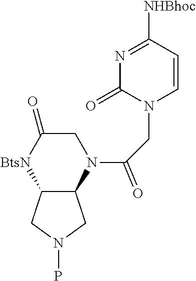 |
| Compound 54c (P = Alloc) Compound 55c (P = Boc) | 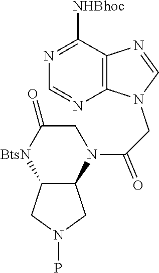 |
| Compound 54d (P = Alloc) Compound 55d (P = Boc) | 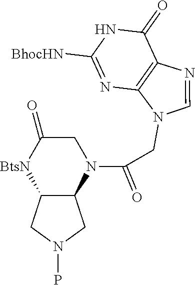 |
| Compound 54e (P = Alloc) Compound 55e (P = Boc) | 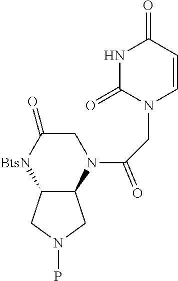 |
| Compound 59a | 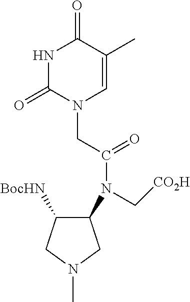 [SMILES: Cc1cn(CC(=O)N(CC(=O)O)[C@H]2CN(C)C[C@@H]2NC(=O)OC(C)(C)C)c(=O)[nH]c1=O] |
| Compound 59b | 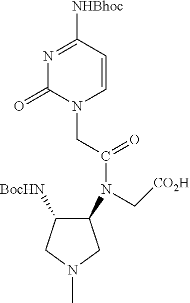 |
| Compound 59c | 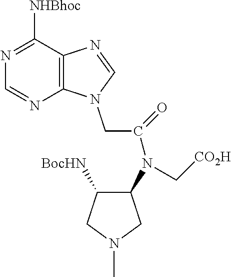 |
| Compound 59d | 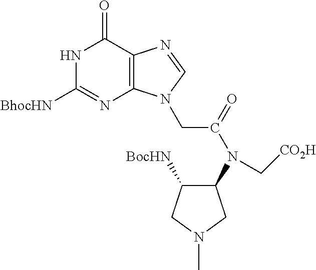 |
| Compound 59e | 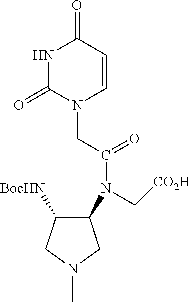 [SMILES: CN1C[C@H](NC(=O)OC(C)(C)C)[C@@H](N(CC(=O)O)C(=O)Cn2ccc(=O)[nH]c2=O)C1] |
| Compound 64a | 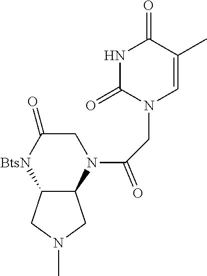 |
| Compound 64b | 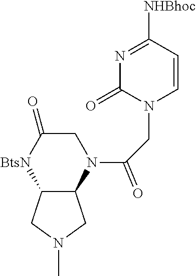 |
| Compound 64c | 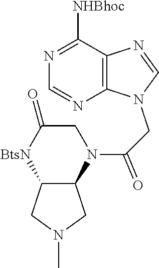 |
| Compound 64d | 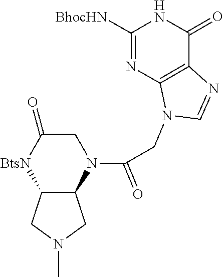 |
| Compound 64e | 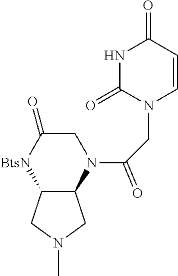 |

Table 1 above shows the PNA monomer derivatives in the present disclosure.

In one embodiment, the invention provides a method of improving solubility and/or nucleic acid affinity of a peptide nucleic acid (PNA) including: incorporating one or more cyclic structural moieties to a PNA monomer.

By “improving”, it is meant that the modification performed to the PNA increases or ameliorates the solubility and/or affinity of said modified PNA (e.g., by the incorporation of one or more cyclic structural moieties) as compared to a PNA that is not modified (e.g., that does not include one or more cyclic structural moieties).

As used herein, “solubility” of the PNA refers to the property of the PNA (or solute) to dissolve in a solvent. The solubility of a solute fundamentally depends on the physical and chemical properties of the solute and solvent as well as on temperature, pressure and presence of other chemicals (including changes to the pH) of the solution. The extent of the solubility of a substance in a specific solvent is measured as the saturation concentration, where adding more solute does not increase the concentration of the solution and begins to precipitate the excess amount of solute. In many aspects, the PNA solvent is a liquid, such as water, a buffer solution, or a physiological solution, and the solubility of the PNA is the solvent can be referred as the water-, buffer-, or physiological solution solubility of the PNA.

As used herein, the term “affinity” is a measure of the tightness with a particular ligand (e.g., an HA polypeptide) binds to its partner (e.g., an HA receptor). Affinities can be measured in different ways. In some aspects, affinity is measured by a quantitative assay (e.g., glycan binding assays). The binding partner concentration (e.g., HA receptor, glycan, etc.) may be fixed to be in excess of ligand (e.g., an HA polypeptide) concentration so as to mimic physiological conditions (e.g., viral HA binding to cell surface glycans). Alternatively, or additionally, in other aspects, binding partner (e.g., HA receptor, glycan, etc.) concentration and/or ligand (e.g., an HA polypeptide) concentration may be varied. In such aspects, affinity (e.g., binding affinity) may be compared to a reference (e.g., a wild-type HA that mediates infection of a humans) under comparable conditions (e.g., concentrations). The methods described herein can improve the affinity of the PNA agents to nucleic acids. The nucleic acid can be an RNA or a DNA.

In some aspects, the one or more cyclic structural moieties comprise tetrahydrofuran, pyrrolidinium, pyrrolidine, or N-methyl pyrrolidine. In other aspects, the tetrahydrofuran, pyrrolidinium, pyrrolidine, or N-methyl pyrrolidine are incorporated into C2-C3 position.

In another embodiment, the invention provides a pharmaceutical composition comprising the PNA with a structure according to formula (II) and a pharmaceutically acceptable carrier. These PNA analogues may be inserted within a standard aegPNA oligomer intermittently solely or in combination with other PNA analogies or be made as a homo-oligomer.

Described herein are PNAs. As used herein, the term “PNA” can be used interchangeably with the terms “PNA agent”, “PNA molecule”, “PNA monomer” and the like without any difference in the meaning, and refer to nucleic acid-based therapeutic agents, and derivatives thereof, produced by the methods described herein.

The term “nucleic acid-based therapeutic agent” as used herein refers to three classes of compounds. The term also includes pharmaceutically acceptable salts, esters, prodrugs, codrugs, and protected forms of the compounds, analogs and derivatives described below. The first class, referred to herein collectively as “antisense nucleic acids,” comprises nucleic acids, preferably oligomers of about 50 monomer units or fewer, which have the ability to hybridize in a sequence-specific manner to a targeted single-stranded RNA or DNA molecule. Members of this class include ordinary DNA and RNA oligomers, DNA and RNA having modified backbones, including but not limited to phosphorothioates, phosphorodithioates, methylphosphonates, and peptide nucleic acids (PNAs), 2′-deoxy derivatives, and nucleic acid oligomers that feature chemically modified purine and pyrimidine bases, or have been lipophilically modified and/or PEGylated to modify their pharmacodynamics. Oligomers that serve as precursors for such agents, such as hairpin RNAs that are converted to siRNAs within cells, are also considered to be within this class. The second class of nucleic acid-based therapeutic agents is aptamers. Aptamers comprises nucleic acids, preferably oligomers of about 50 monomer units or fewer, which have the ability to bind with structural specificity to a non-oligonucleotide target molecule, or to an oligonucleotide in a manner other than through sequence-specific hybridization. Members of this class include DNA and RNA aptamers, and modifications thereof including but not limited to mirror-image DNA and RNA (“Spiegelmers”), peptide nucleic acids, and nucleic acid oligomers that have otherwise been chemically modified as described above. Again, any of these species may also feature chemically modified purines and pyrimidines or may be lipophilically modified and/or PEGylated (see M. Rimmele, Chembiochem. 4: 963-71 (2003); and A. Vater and S. Klussmann, Curr. Opin. Drug Discov. Devel. 6: 253-61 (2003), for recent reviews of aptamer technology). It will be appreciated that many members of this second class will, in addition to their structure-specific affinity for the target molecule, have sequence-specific affinity for a putative DNA or RNA sequence. The third class of nucleic acid-based therapeutic agents, referred to herein as “nucleic acid enzymes.” comprises nucleic acids that are capable of recognizing and catalyzing the cleavage of target RNA molecules, in a sequence-specific manner. The class includes hammerhead ribozymes, minimized hammerheads (“minizymes”), ‘10-23’ deoxyribozymes (“DNAzymes”), and the like. As with antisense and aptamer molecules, the class includes catalytic species that have been chemically modified.

PNA is a totally artificial molecule that is used as a DNA analog in genetic engineering and consisting of a polypeptide backbone with nucleic acid bases attached as side chains. The polypeptide backbone of PNA is not identical to that of natural proteins as it is designed to space out the bases that it carries at the same distances as found in genuine nucleic acids. This enables a strand of PNA to base pair with a complementary strand of DNA or RNA. In PNAs the phosphodiester backbone of DNA molecules is replaced by repetitive units of N-(2-aminoethyl) glycine to which the purine and pyrimidine bases are attached via a methyl carbonyl linker. The procedures for PNA synthesis are similar to those employed for peptide synthesis, using standard solid-phase manual or automated synthesis. By convention, PNAs are depicted like peptides, with the N-terminus at the left (or at the top) position and the c-terminus at the right (or at the bottom) position. PNAs hybridize to complementary DNA or RNA sequences in a sequence-dependent manner, following the Watson-Crick hydrogen bonding scheme. PNAs can bind to complementary nucleic acids in both parallel and antiparallel orientation. However, the antiparallel orientation illustrated in this figure is preferred.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e. transfection of, cells, in particular, in the form of RNA which can be prepared by in vitro transcription from a DNA template. The RNA can moreover be modified before application by stabilizing sequences, capping, and polyadenylation.

As used herein, term “amino acid” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

PNAs can be designed to be complementary to a target DNA or RNA sequence of interest.

By “complementarity”, it is meant that the sequence of the nucleobases of the PNA form interactions with the nucleobases on the target DNA or RNA. The nucleobases can be natural (primary or canonical), modified, and/or artificial nucleobases. Primary nucleobases include adenine (A) and guanine (G), which are purines, and thymine (T), cytosine (C) and uracil (U), which are pyrimidines. Modified nucleobases are non-canonical bases and include 5-methylcytosine (m⁵C), pseudouridine (Y), dihydrouridine (D), inosine (I), 7-methylguanosine (m⁷G), hypoxanthine and xanthine. Artificial nucleobases include nucleobase analogs such as aminoallyl, isoguanine, isocytosine or the fluorescent 2-amino-6-(2-thienyl)purine and pyrrole-2-carbaldehyde for example. Purines are larger than pyrimidines. Both types of molecules complement each other and can only base pair with the opposing type of nucleobase. In nucleic acid, nucleobases are held together by hydrogen bonding, which only works efficiently between adenine and thymine and between guanine and cytosine. The base complement A=T or A=U shares two hydrogen bonds, while the base pair G=C has three hydrogen bonds. All other configurations between nucleobases would hinder double helix formation. DNA strands are oriented in opposite directions, they are said to be antiparallel. A complementary strand of DNA or RNA may be constructed based on nucleobase complementarity.

The PNA described herein includes from about 13 to 30 nucleobases. For example, the PNA can include 13, 14, 15, 16, 17, 18, 19, or 20 nucleobases.

The PNA described herein has a complementarity that is at least 75% to the target DNA or RNA sequence. For example, the PNA can have 75, 80, 85, 90, 91, 92, 93, 94, 95, 96, 97, 98, 99% or more complementarity to the sequence of the target DNA or RNA.

Chemical modifications in PNA have been done by introducing different substituents, conformationally constrained cyclic backbones, or modified nucleobases. For instance, the presence of amide group provides rigidity to the structure, which was replaced with an amine group, resulting into the reduction of binding affinity which further confirmed the significance of ‘constrained flexibility’ of PNA backbone. In order to increase rigidity, a cyclic group such as cyclohexyl was incorporated but it resulted in decreased binding affinity. On the contrary, introduction of cyclopentyl, (2S,5R)-aminoethyl pipecolyl, prolyl derivatives were found to improve binding affinity. Furthermore, charged PNAs have been developed to enhance the solubility and cellular delivery by introducing groups like phosphates and guanidium in the backbone. The PNA described herein includes modification of the backbone to provide for more soluble PNA variants.

The PNAs described herein can be formulated in a pharmaceutical composition.

As used herein, the term “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. Examples of active ingredient include, but are not limited to, chemical compound, drug, therapeutic agent, small molecule, etc. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino. In certain illustrative embodiments, the active agent is a PNA.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO). The term “pharmaceutically acceptable salts” refers to physiologically and pharmaceutically acceptable salts of the compounds of the invention, e.g., salts that retain the desired biological activity of the parent compound and do not impart undesired toxicological effects thereto.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Also disclosed herein are pharmaceutical compositions including compounds with the structures of Formula (II) or any PNA agents based on the monomer derivatives selected from Table 1. Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, drenches (aqueous or non-aqueous solutions or suspensions), boluses, granules, pastes for application to the tongue, sterile solution or suspension, cream, ointment, pessary, foam, etc.

Methods of preparing formulations or compositions comprising described compounds include a step of bringing into association a compound of the present disclosure with the carrier and, optionally, one or more accessory ingredients. In general, formulations may be prepared by uniformly and intimately bringing into association a compound of the present disclosure with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming micro-encapsuled matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/orantifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient.

In an additional embodiment, the invention provides a method of reducing expression of a target gene in a cell including: contacting a cell in which the target is expressed with a PNA agent with a structure according to formula (II).

The PNA described herein can target any DNA or RNA sequence. In preferred aspects, the PNA targets a gene, or a mRNA encoding by a gene, and does not target a promoter region of a gene.

As used herein, the term “gene” has the same general meaning as understood in the art. In some embodiments, the term “gene” includes gene regulatory sequences (e.g., promoters, enhancers, etc.) and/or intron sequences. In some embodiments, the term refers to nucleic acids that do not encode proteins but rather encode functional RNA molecules such as RNAs, RNAi-inducing agents, etc. Alternatively, or additionally, in some embodiments, the term “gene”, as used in the present application, refers to a portion of a nucleic acid that encodes a protein. Whether the term encompasses other sequences (e.g., non-coding sequences, regulatory sequences, etc.) will be clear from context to those of ordinary skill in the art.

As used herein the term “gene product” or “expression product” refers to an RNA transcribed from the gene (pre- and/or postprocessing) or a polypeptide (pre- and/or post-modification) encoded by an RNA transcribed from the gene.

In various aspects, the PNA targets a DNA or RNA that carries a mutation, such as a mutation that is associated with or responsible for the malignant transformation of a cell, or the development of cancer in a subject. The “mutant” refers to any alteration in a nucleic acid (or optionally genetic) sequence compared to its naturally occurring counterpart. Mutant may also refer to the gene product (such as a protein), cells, or organism that possesses the mutated gene. Nucleic acid sequences possessing mutations can also be referred to as mutant sequence elements.

In other aspects, the PNA targets an oncogene. As used herein, the term “oncogene” refers to those genes whose products are associated with causing cancer, dysplasia, hyperplasia, etc. in an organism. Non-limiting example of oncogenes that can be targeted by the PNA of the invention include, but are not limited to: ABL1, ABL2, ALK, AKT1, AKT2, ATF1, BCL11A, BCL2, BLC3, BCL6, BCR, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFRIOP, FGFR2, FUS, GOLGA5, HMGA1, HMGA2, HRAS, IRF4, IDHI, IDH2, JUN, KIT, KRAS, LCK, LM02, MAF, MAFB, MAML2, MDM2, MET, MITF, MLL, MPL, MYB, MYC, MYCLI, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SSI 8, TCL1A, TET2, TFG, TLX1, TPR, and USP6.

In some aspects, PNA agents target a site including or consisting of a region including an amplification of a gene. In various, PNA agents target gene amplifications including AKT2. CDK4, MDM2, MYCN, CCNE, CCND1, KRAS, HRAS, EGFR, ERBB2, ERBB1, FGF, FGFR1. FGFR2, MYC, MYB, and MET.

In various aspects, the PNA described herein reduces the expression of the target gene in a cell. By “reducing the expression” of the gene, it is meant that the PNA can reduce or inhibit the transcription of the gene (binding of the PNA to a DNA target) and/or that the PNA can reduce of inhibit the translation of the mRNA (binding of the PNA to a RNA target). The reduction of the level of expression can be measured by any means known in the art to evaluate gene expression, such as PCR, qPCR, RT-PCR, RT-qPCR, western blot, immunofluorescence, immunohistochemistry, and the like.

A reduction in the expression of a gene of interest can be evaluated by measuring the expression of the gene prior to contacting the cell with a PNA, and after contacting the cell with a PNA, and comparing the expression levels. An expression level measured after contacting a cell with a PNA that is lower than an expression level measured prior to contacting the cell with the PNA is a reduced expression level.

In some aspects, the PNA reduces the expression of the target gene by at least 10%. For example, the PNA can reduce gene expression by 10, 20, 30, 40, 50, 60, 70, 80, 90, 95% or more, as compared to the expression level of the gene in a cell that is not contacted with the PNA.

In a further embodiment, the invention provides a method for identifying and/or characterizing PNA agents for target inhibition including: contacting a system in which a target is expressed with a PNA agent with a structure according to formula (II); determining a level or activity of the target in the system when the PNA agent is present as compared with a target reference level or activity observed under otherwise comparable conditions when it is absent; and classifying the PNA agent as a target inhibitor if the level or activity of the target is significantly reduced when the PNA agent is present as compared with the target reference level or activity.

As used herein, a “system” can refer to any in vitro or in vivo system, which can be contacted with a PNA described herein to assess the inhibition ability of the PNA. A system can be a cell or an organism. For example, a cell can be an eukaryotic cell, such as CHO, COS (e.g. COS-7), 3T3-F442A, HeLa, HUVEC, HUAEC, NIH 3T3, Jurkat, 293, 293H, 293F, or any other eukaryotic cell such as a human cell, or a cancer cell. An organism can be an animal, such as a rodent, or a primate.

The PNAs described herein, or the pharmaceutical compositions including the PNAs described herein can be administered to subjects in need thereof, for example for the treatment of cancer in the subject.

In some aspects, methods for treating or reducing the risk of a disease, disorder, or condition including: administering to a subject susceptible to the disease, disorder, or condition PNA agents are provided. In various aspects, the disease or disorder is cancer. In some aspects, the cancer is selected from melanoma, ocular melanoma and/or sarcoma.

In one embodiment, the present invention provides a method for treating cancer in a subject including administering a PNA agent with a structure of formula (II).

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well by infusion, inhalation, and nebulization.

The terms “therapeutically effective amount”, “effective dose,” “therapeutically effective dose”, “effective amount,” or the like refer to that amount of the subject PNA that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of cancer). Such amount should be sufficient to lessen or reduce symptoms associated with the cancer, or limit or reduce its development, progression, and/or recurrence. The effective amount can be determined as described herein.

The term “cancer” refers to a group diseases characterized by the abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, the term “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, stomach, biliary, colon, rectal, liver, kidney, lung, testicular, breast, ovarian, pancreatic, brain, cervical and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma VMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

As used herein, the “treating cancer” refers to the ability of the PNAs described herein to reduce or inhibit the level or activity of a target DNA or mRNA. In some aspects, inhibiting or reducing the level or activity of the target includes inhibit or reduce cancer cell viability. In various aspects, a significant reduction in the level or activity of the target corresponds to a greater than 50% decrease in cancer cell viability. In some aspects, complete suppression of gene expression is not necessary for significant suppression of cancer cell proliferation/decreasing cell viability. In other aspects, inhibiting or reducing the level or activity of the target includes increasing survival of the organism. In some embodiments, a significant reduction in the level or activity of the target includes a greater than 50% increase in survival of the organism.

The dose of PNA agent of the present disclosure to be administered to a subject can optionally range from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other aspects, the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg. 100 mg, 150 mg. 200 mg. 250 mg and 300 mg. The progress of therapy is monitored by conventional techniques and assays.

In some aspects, a PNA agent of the present disclosure can be administered to a human patient at an effective amount (or dose) of less than about 1 μg/kg, for instance, about 0.35 to about 0.75 μg/kg or about 0.40 to about 0.60 μg/kg. In some aspects, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various aspects, the absolute dose of an agent is about 2 μg/subject to about 45 μg/subject, or about 5 to about 40, or about 10 to about 30, or about 15 to about 25 μg/subject. In some aspects, the absolute dose of an agent is about 20 μg, or about 30 μg, or about 40 μg.

In various aspects, the dose of a PNA agent of the present disclosure may be determined by the human patient's body weight. For example, an absolute dose of an agent of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain aspects, a PNA agent in accordance with the methods provided herein is administered subcutaneously (s.c.), intravenously (i.v.), intramuscularly (i.m.), intranasally or topically. Administration of an agent described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to about 3 times (e.g. 1, or 2 or 3 times).

In certain aspects, the treatment method further includes administering to the subject an anti-cancer treatment.

The term “anti-cancer therapy” or “anti-cancer treatment” as used herein is meant to refer to any treatment that can be used to treat cancer, such as surgery, radiotherapy, chemotherapy, immunotherapy, targeted therapy, checkpoint inhibitor therapy, and any combination thereof.

The term “chemotherapeutic agent” as used herein refers to any therapeutic agent used to treat cancer. Examples of chemotherapeutic agents include, but are not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

The term “immunotherapy” refers to any type of therapy that includes modulating the immune system or the immune response. Modulating the immune system includes inducing, stimulating or enhancing the immune system as well as reducing, suppressing or inhibiting the immune system. Immunotherapy can be active or passive. Passive immunotherapy relies on the administration of monoclonal antibodies directed against the target to eliminate. For example, tumor-targeted monoclonal antibodies have demonstrated clinical efficacy to treat cancer. Active immunotherapy aims to induce a cellular immunity and establish immunological memory against the target agent. Active immunotherapy includes but is not limited to vaccination, and immune modulators.

Examples of immunotherapy include treatment with antibodies including, but not limited to, alemtuzumab, Avastin® (bevacizumab), Bexxar (tositumomab), CDP 870, and CEA-Scan (arcitumomab), denosumab, Erbitux® (cetuximab), Herceptin® (trastuzumab), Humira® (adalimumab), IMC-IIF 8, LeukoScan® (sulesomab), MabCampath® (alemtuzumab), MabThera® (Rituximab), matuzumab, Mylotarg® (gemtuzumab oxogamicin), natalizumab, NeutroSpec® (Technetium (99mTc) fanolesomab), panitumamab, Panorex® (Edrecolomab), ProstaScint® (Indium-Ill labeled Capromab Pendetide), Raptiva® (cfalizumab), Remicade® (infliximab), ReoPro® (abciximab), rituximab, Simulect® (basiliximab), Synagis® (palivizumab), TheraCIM hR3, tocilizumab, Tysabri® (natalizumab), Verluma® (nofetumomab), Xolair® (omalizumab), Zenapax® (dacliximab), Zevalin® (ibritumomab tiuxetan (IDEC-Y2B8) conjugated to yttrium 90), Gilotrif® (afatinib), Lynparza® (olaparib), Perjeta® (pertuzumab), Opdivo® (nivolumab), Bosulif® (bosutinib), Cabometyx® (cabozantinib), trastuzumab-dkst (Ogivri), Sutent® (sunitinib malate), Adcetris® (brentuximab vedotin), Alecensa® (alectinib), Calquence® (acalabrutinib), Yescarta® (ciloleucel), Verzenio® (abemaciclib), Keytruda® (pembrolizumab), Aliqopa® (copanlisib), Nerlynx® (neratinib), Imfinzi® (durvalumab), Darzalex® (daratumumab), Tecentriq® (atezolizumab), and Tarceva® (crlotinib).

“Checkpoint inhibitor therapy” is a form of cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

There are several checkpoint inhibitors that are currently used to treat cancer. PD-1 inhibitors include Pembrolizumab (Keytruda®) and Nivolumab (Opdivo®). PD-L1 inhibitors include Atezolizumab (Tecentriq®), Avelumab (Bavencio®) and Durvalumab (Imfinzi®). CTLA-4 inhibitors include Iplimumab (Yervoy®). There are several other checkpoint inhibitors being developed including an anti B7-H3 antibody (MGA271), an anti-KIR antibody (Lirilumab) and an anti-LAG3 antibody (BMS-986016).

The PNA agent can be administered prior to, simultaneously with or following the administration of the anti-cancer treatment.

In some aspects, administration of the PNA of the invention can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the PNA described herein to a subject can be in combination with an anti-cancer treatment. Such therapies can be administered prior to, simultaneously with, or following administration of the composition of the present invention.

In some aspects, the PNA agent can be administered orally, parenterally, intradermally, transdermally, or by inhalation.

PNA agents can be created complementary to any gene of interest, and therefore have multiple applications. Targeting and binding by PNA agents would have uses as research tools, medical diagnostics and pharmaceutical treatments. In some aspects. PNA agents can be used to target and bind specific genetic sequences. In other aspects, PNA agents can be used to suppress expression of genetic sequences. PNA agents targeted to specific genes can serve as valuable research tools in understanding the function of those genes. Suppressing the expression of particular gene products would help elucidate and discover the role of those products in different biological pathways.

In some embodiments, the present disclosure provides a variety of kits for conveniently and/or effectively carrying out methods in accordance with the present invention. Kits typically comprise one or more PNA agents. In some aspects, kits for use in accordance with the present disclosure may include one or more reference samples; instructions (e.g., for processing samples, for performing tests, for interpreting results, for administering PNA agents, for storage of PNA agents, etc.); buffers; and/or other reagents necessary for performing tests. In other aspects, kits can comprise panels of PNA agents. Other components of kits may include cells, cell culture media, tissue, and/or tissue culture media.

In various aspects, kits include a number of unit dosages of a pharmaceutical composition comprising PNA agents. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may include one or more vessels or containers so that certain of the individual components or reagents may be separately housed. Kits may comprise a means for enclosing the individual containers in relatively close confinement for commercial sale, e.g., a plastic box, in which instructions, packaging materials such as styrofoam, etc., may be enclosed. In some aspects, kits are used in the treatment, diagnosis, and/or prophylaxis of a subject suffering from and/or susceptible to cancer or other disorder. In other aspects, such kits comprise (i) at least one PNA agent; (ii) a syringe, needle, applicator, etc. for administration of the at least one PNA agent to a subject; and (iii) instructions for use.

Presented below are examples discussing PNA monomer derivatives and the PNAs contemplated for the discussed applications. The following examples are provided to further illustrate the embodiments of the present disclosure but are not intended to limit the scope of the disclosure. While they are typical of those that might be used, other procedures, methodologies, or techniques known to those skilled in the art may alternatively be used.

### EXAMPLES

### Example 1

### Synthesis of Boc-Protected Tetrahydrofuran-Based PNA Monomer Derivatives (Compounds 5a to 5e)

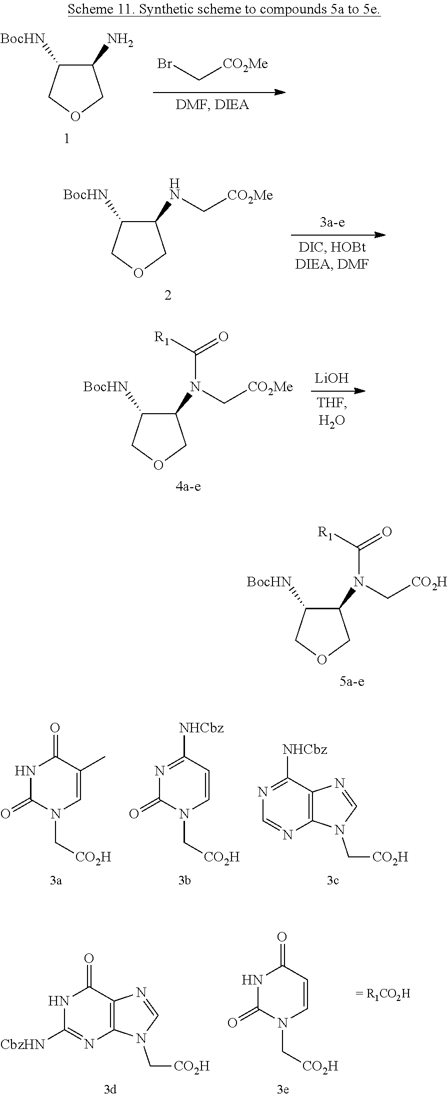

Scheme 11 shows the synthetic procedure to obtain compounds 5a to 5e, Boc-protected tetrahydrofuran-based PNA monomer derivatives, according to some embodiments of the present disclosure. The starting material, compound 1, in this synthetic route is commercially available (—from Entrechem). ICH₂CO₂Me can be replaced with BrCH₂CO₂-allyl for use with Fmoc-derivatives as alkaline ester hydrolysis may be replaced with better orthogonal conditions, Pd(PPh₃)₄ for alloc deprotection.

### Example 2

### Synthesis of Fmoc-Protected Tetrahydrofuran-Based PNA Monomer Derivatives (Compounds 10a to 10e)

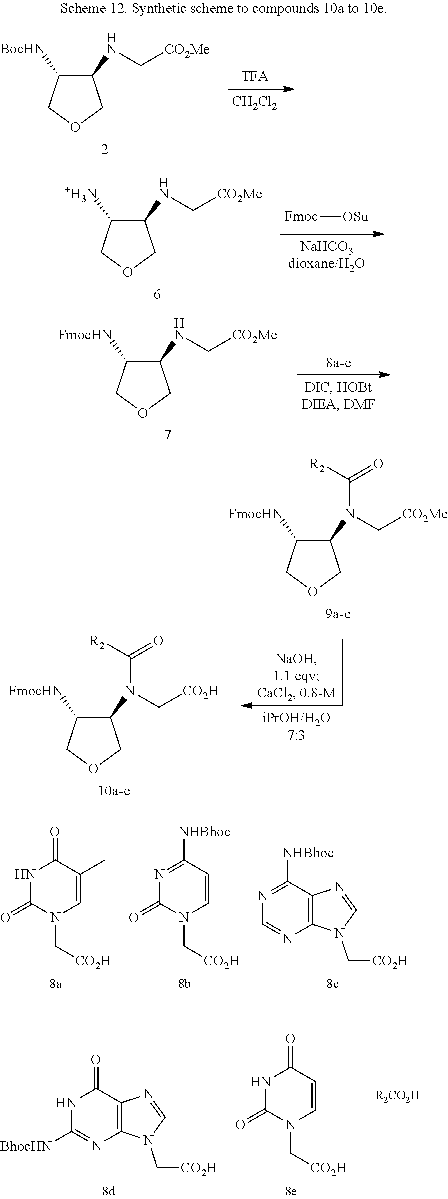

Scheme 12 shows the synthetic procedure to obtain compounds 10a to 10e, Fmoc-protected tetrahydrofuran-based PNA monomer derivatives, according to some embodiments of the present disclosure. The ester-selective hydrolysis reaction in the final step can be found in Pascal, et al. Tet. Lett., (1998) 39, 5031-34.

### Example 3

### Synthesis of Bts-Protected Tetrahydrofuran-Based PNA Monomer Derivatives (Compounds 14a to 14e)

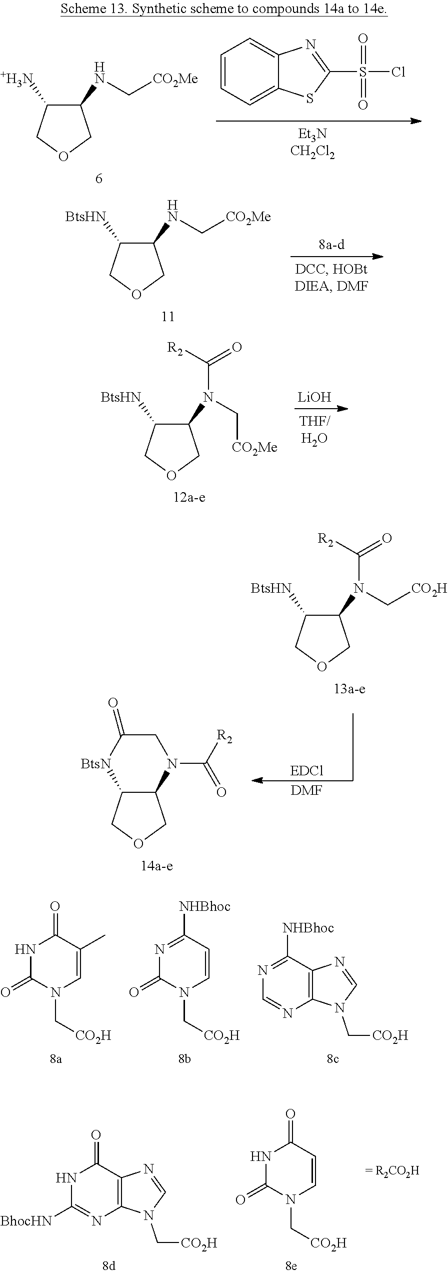

Scheme 13 shows the synthetic procedure to obtain compounds 14a to 14e, Bts-protected tetrahydrofuran-based PNA monomer derivatives, according to some embodiments of the present disclosure. The synthesis is generally based on Lee, et al. (2007) Org. Lett., 9:3291-93.

### Example 4

### Synthesis of Boc-Protected Pyrrolidinium-Based PNA Monomer Derivatives (Compounds 20a to 20e)

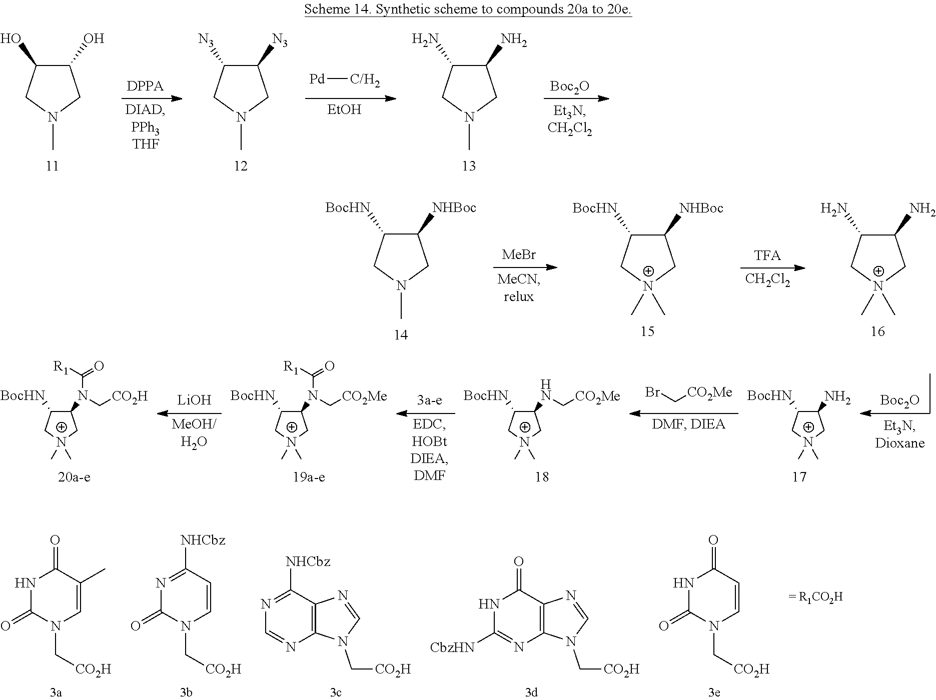

Scheme 14 shows the synthetic procedure to obtain compounds 20a to 20e, Boc-protected pyrrolidinium-based PNA monomer derivatives, according to some embodiments of the present disclosure.

### Example 5

### Synthesis of Fmoc-Protected Pyrrolidinium-Based PNA Monomer Derivatives (Compounds 22a to 22e)

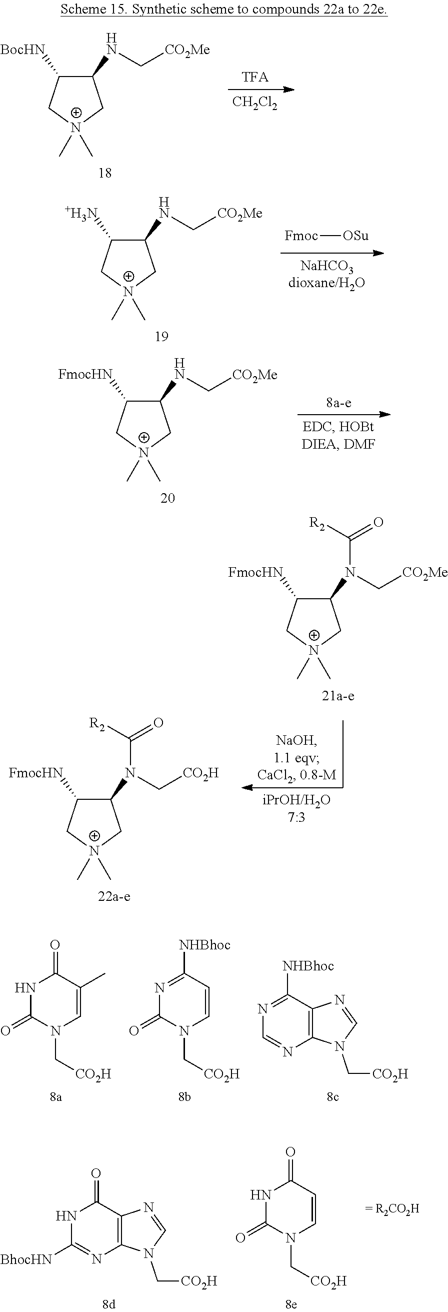

Scheme 15 shows the synthetic procedure to obtain compounds 22a to 22e, Fmoc-protected pyrrolidinium-based PNA monomer derivatives, according to some embodiments of the present disclosure. ICH₂CO₂Me can be replaced with BrCH₂CO₂-allyl for use with Fmoc-derivatives as alkaline ester hydrolysis may be replaced with better orthogonal conditions, Pd(PPh₃)₄ for Alloc deprotection.

### Example 6

### Synthesis of Bts-Protected Pyrrolidinium-Based PNA Monomer Derivatives (Compounds 32a to 32e)

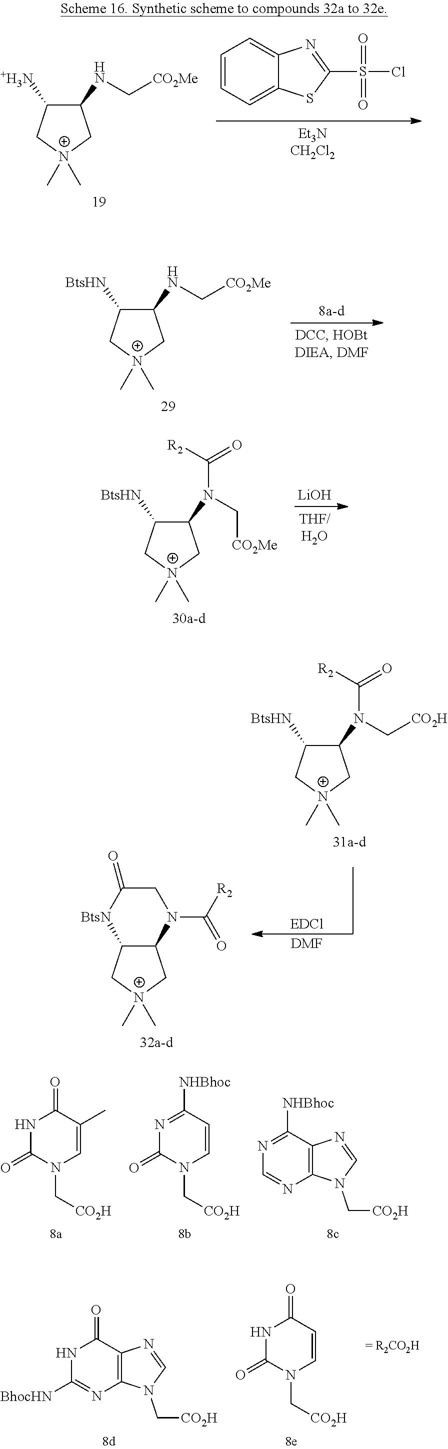

Scheme 16 shows the synthetic procedure to obtain compounds 32a to 32e, Fmoc-protected pyrrolidinium-based PNA monomer derivatives, according to some embodiments of the present disclosure. The synthesis is generally based on Lee, et al. (2007) Org. Lett., 9:3291-93.

### Example 7

### Synthesis of Boc, Fmoc, and Bts-Protected Pyrrolidine-Based PNA Monomer Derivatives (Compounds 49a-e, 50a-e, 52a-e, 54a-e and 55a-e)

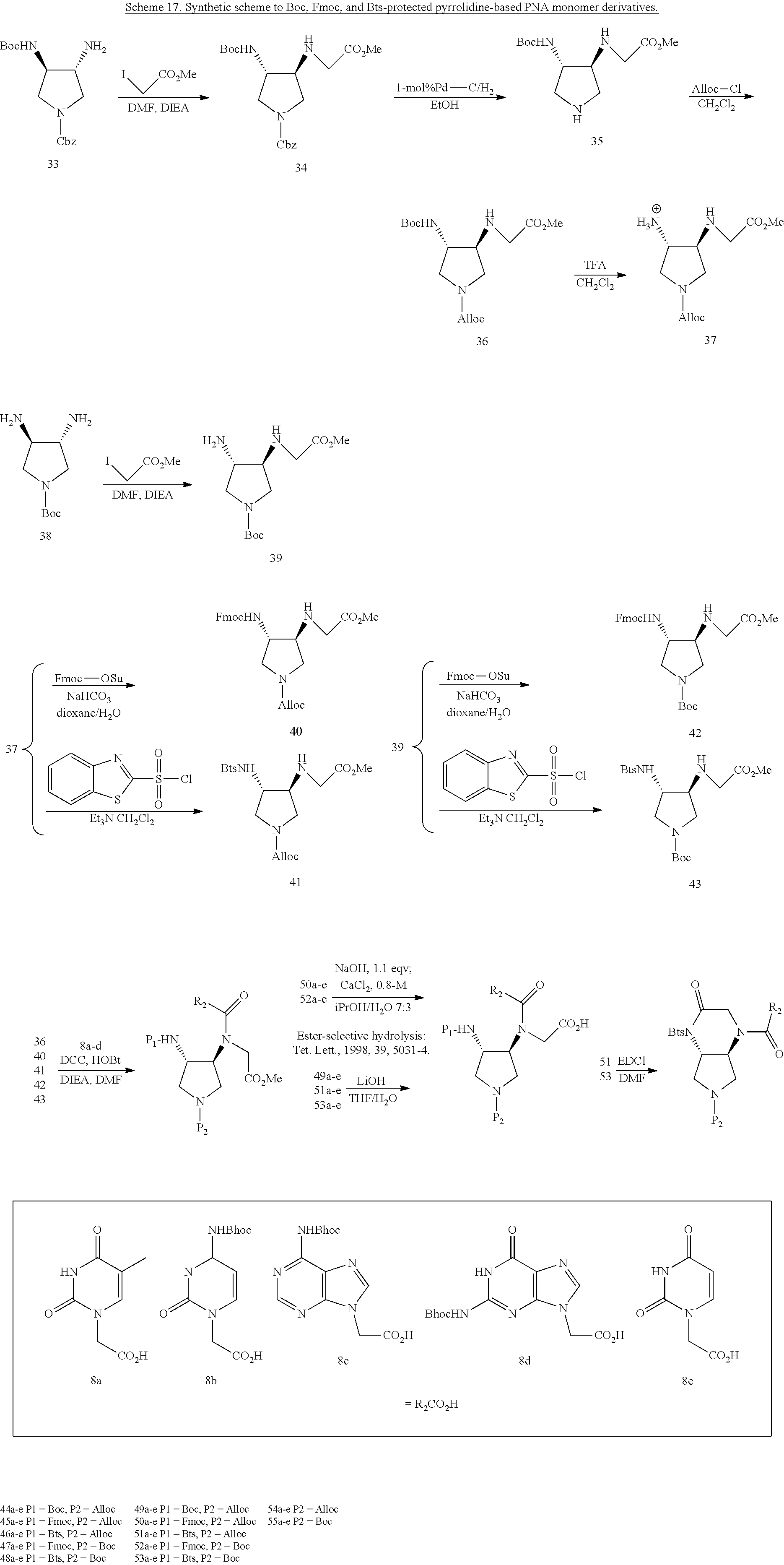

Scheme 17 shows the synthetic procedure to obtain compounds 49a-e (Boc-protected pyrrolidine-based PNA monomer derivatives), 50a-e and 52a-e (Fmoc-protected pyrrolidine-based PNA monomer derivatives), 54a-e and 55a-e (Bts-protected pyrrolidine-based PNA monomer derivatives), according to some embodiments of the present disclosure. ICH₂CO₂Me can be replaced with BrCH₂CO₂-allyl for alkylation of 38 for use with Fmoc-derivatives as alkaline ester hydrolysis may be replaced with better orthogonal conditions, Pd(PPh₃)₄ for Alloc deprotection.

### Example 8

### Synthesis of Boc-Protected N-methyl Pyrrolidine-Based PNA Monomer Derivatives (Compounds 59a to 59e)

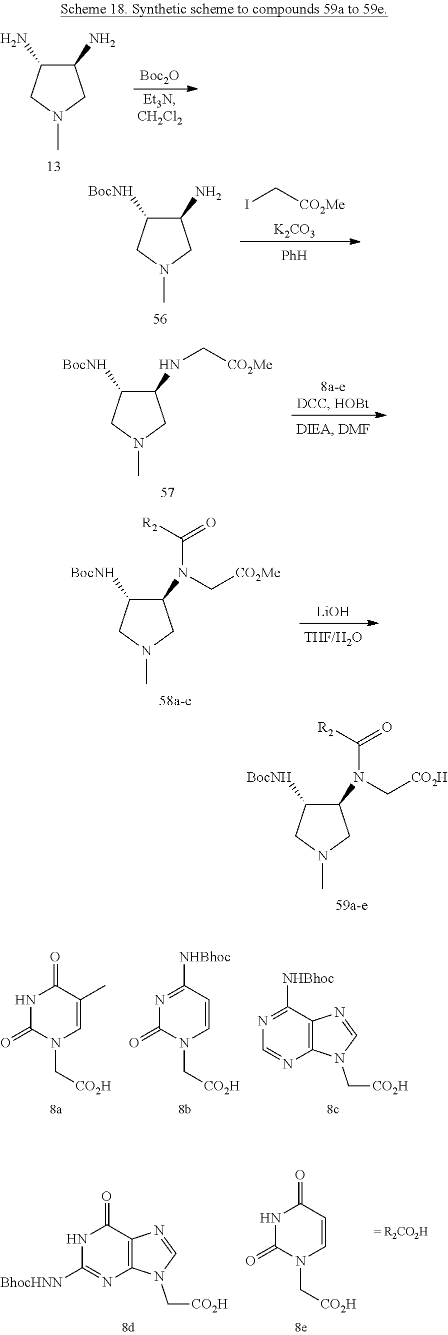

Scheme 18 shows the synthetic procedure to obtain compounds 59a to 59e, Boc-protected N-methyl pyrrolidine-based PNA monomer derivatives, according to some embodiments of the present disclosure.

### Example 9

### Synthesis of Bts-Protected N-methyl Pyrrolidine-Based PNA Monomer Derivatives (Compounds 64a to 64e)

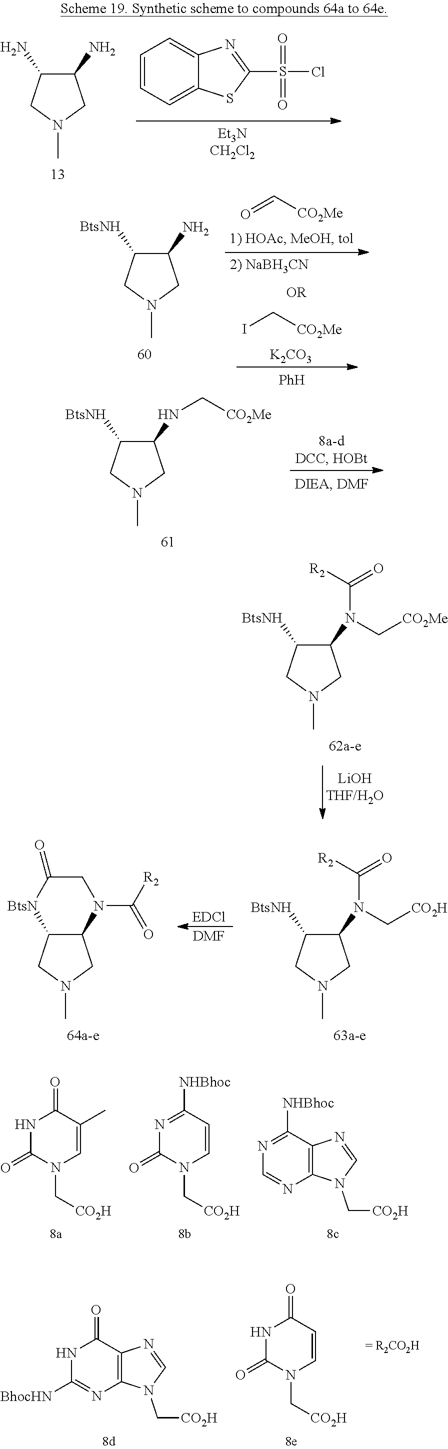

Scheme 19 shows the synthetic procedure to obtain compounds 64a to 64e, Bts-protected N-methyl pyrrolidine-based PNA monomer derivatives, according to some embodiments of the present disclosure.

### Example 10

### Synthesis of Peptide Nucleic Acids (PNAs) Based on the PNA Monomer Derivatives of the Present Disclosure

The solid phase peptide synthesis used to synthesize the PNAs according to the present invention can follow the procedure described in Shaikh, et al. (2020) Methods Mol. Biol. 2105:1-16. which is incorporated herein by reference in its entirety.

Synthesis of tetrahydrofuran, pyrrolidinium, pyrrolidine, and N-methyl pyrrolidine-based PNA monomer derivatives according to the present disclosure are shown in Examples 1-9 above. Other PNA monomer derivatives used in the present study, including PEG-based and guanidine-based PNA monomers can refer to literature, such as Sahu et al. (2009) J. Org. Chem. 74:1509-16 and Sahu et al. (2011) J. Org. Chem. 76:5614-27.

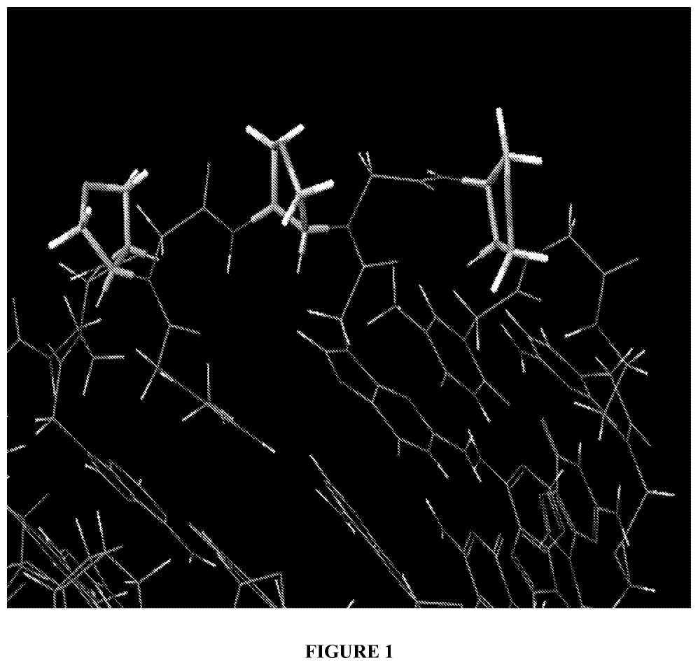

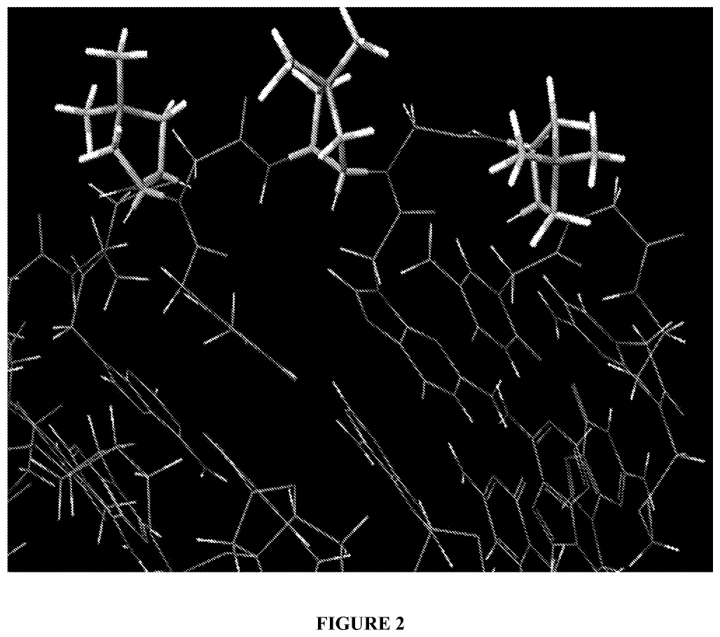

FIG. 1 shows the tetrahydrofuran dihedral clamp 3D structure highlighted against a conventional PNA oligomer. FIG. 2 shows N-Mez pyrrolidinium dihedral clamp 3D structure highlighted against a conventional PNA oligomer.

Although the invention has been described with reference to the above examples, it will be understood that modifications and variations are encompassed within the spirit and scope of the invention. Accordingly, the invention is limited only by the following claims.

1. A compound according to Formula (I) 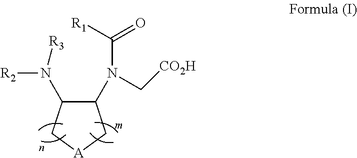
or an optically pure stereoisomer, pharmaceutically acceptable salt, or solvate thereof, wherein
n is an integer selected from 0 to 2;
m is an integer selected from 0 to 2;
A is selected from the group consisting of —O—, —S—, —NR₄—, and 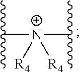
R₁ is selected from the group consisting of 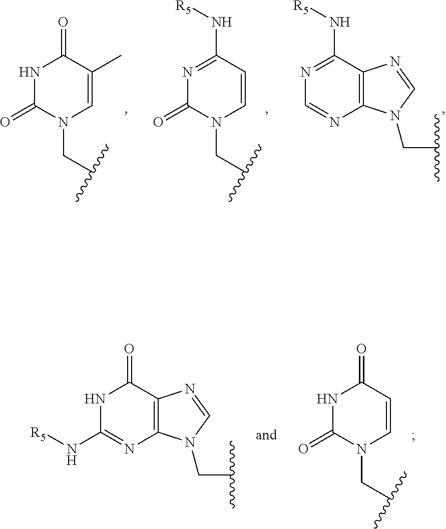
each R₂ and R₅ is independently H or a protective group, the protective group is selected from the group consisting of tert-butyloxycarbonyl (Boc), fluorenylmethoxycarbonyl (Fmoc), benzothiazole-2-sulfonyl (Bts), carboxybenzyl (Cbz), benzhydryloxycarbonyl (Bhoc), p-nitrophenyl, 1-adamantyl formate, allyl formate, triphenylmethyl, benzyl, acetyl, trifluoroacetyl, and p-toluenesulfonyl.
R₃ is H, methyl, ethyl, or propyl; and
each R₄ is independently H, methyl, ethyl, or propyl.

2. The compound of claim 1, wherein n is 1.

3. The compound of claim 1, wherein m is 1.

4. The compound of claim 1, wherein A is —O— or

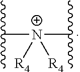

5. The compound of claim 1, wherein R₂ is tert-butyloxycarbonyl (Boc) or fluorenylmethoxycarbonyl protecting group (Fmoc).

6. The compound of claim 1, wherein R₅ is carboxybenzyl (Cbz).

7. The compound of claim 1 selected from the group consisting of

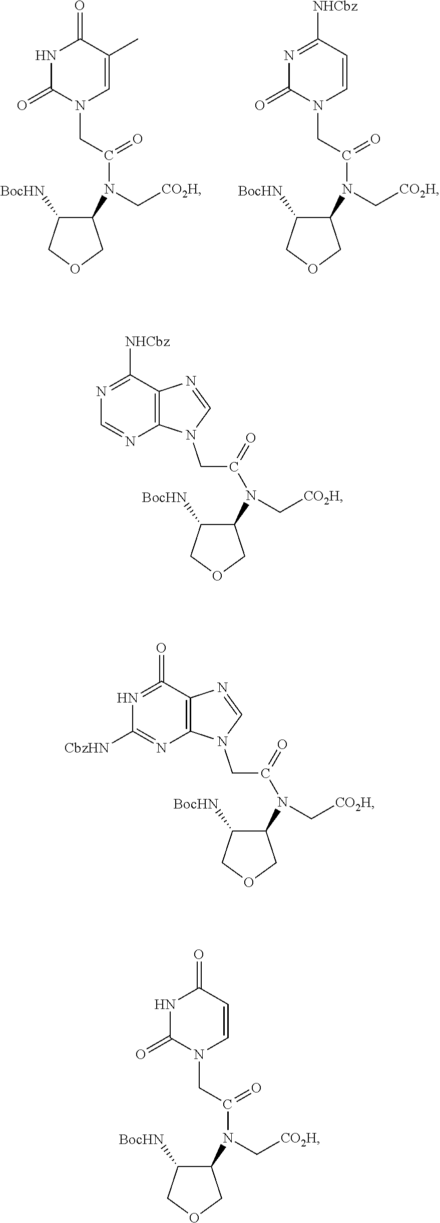

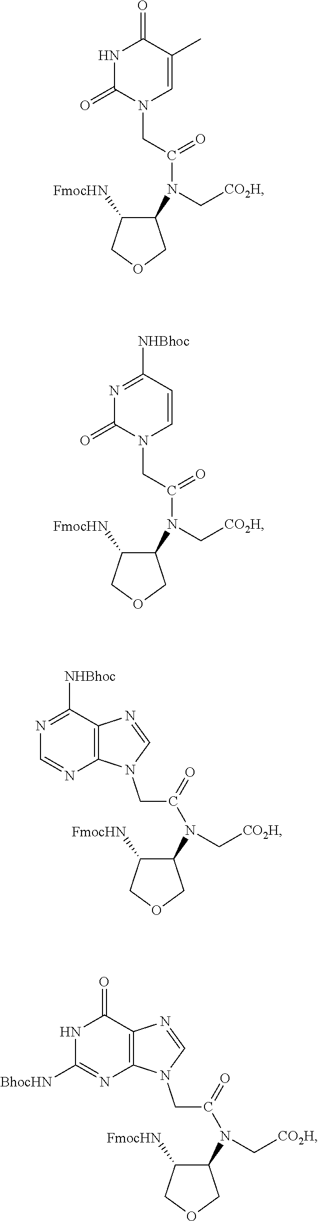

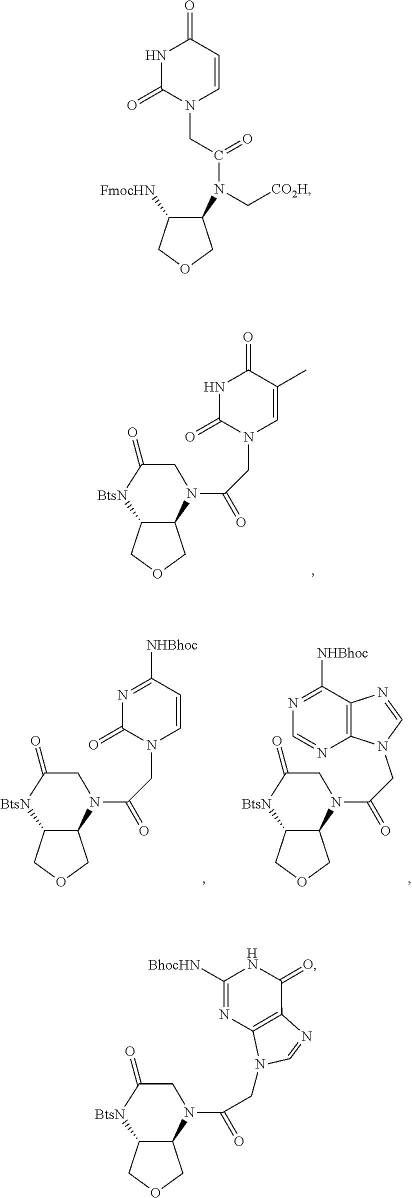

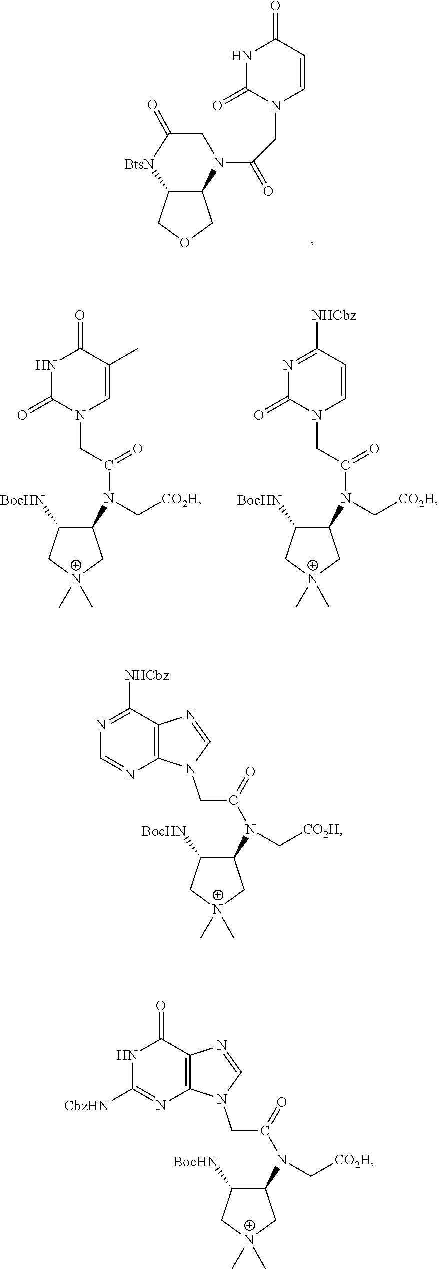

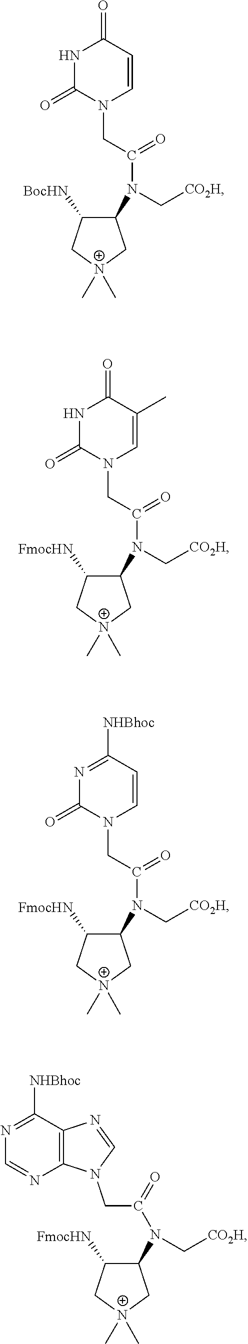

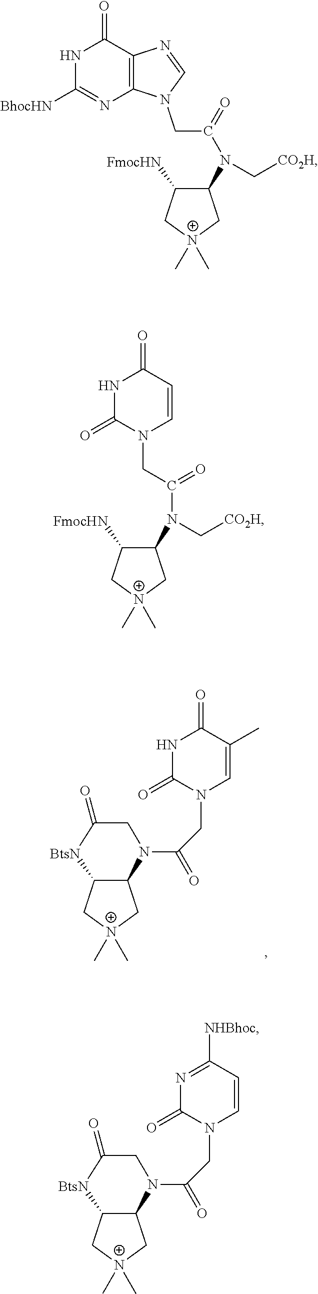

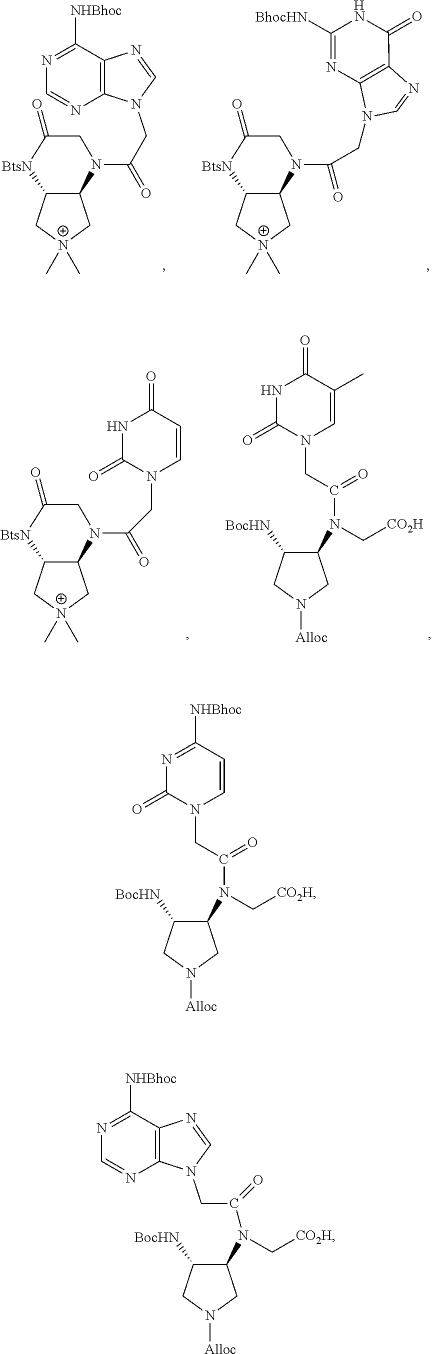

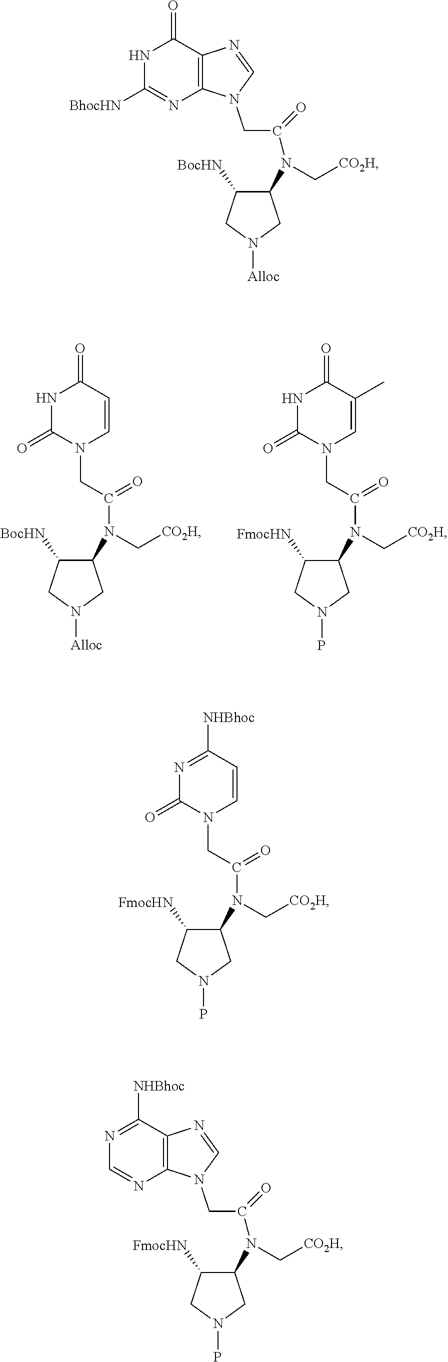

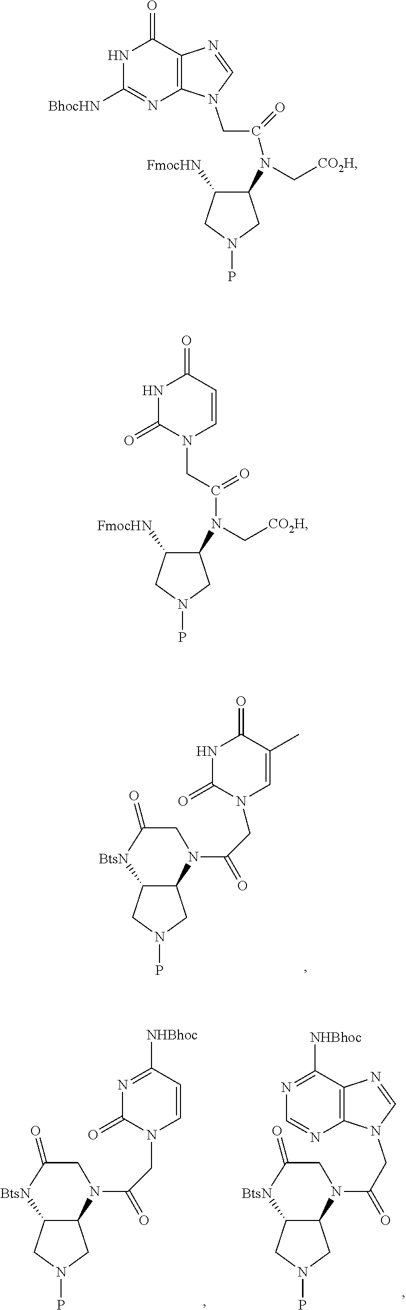

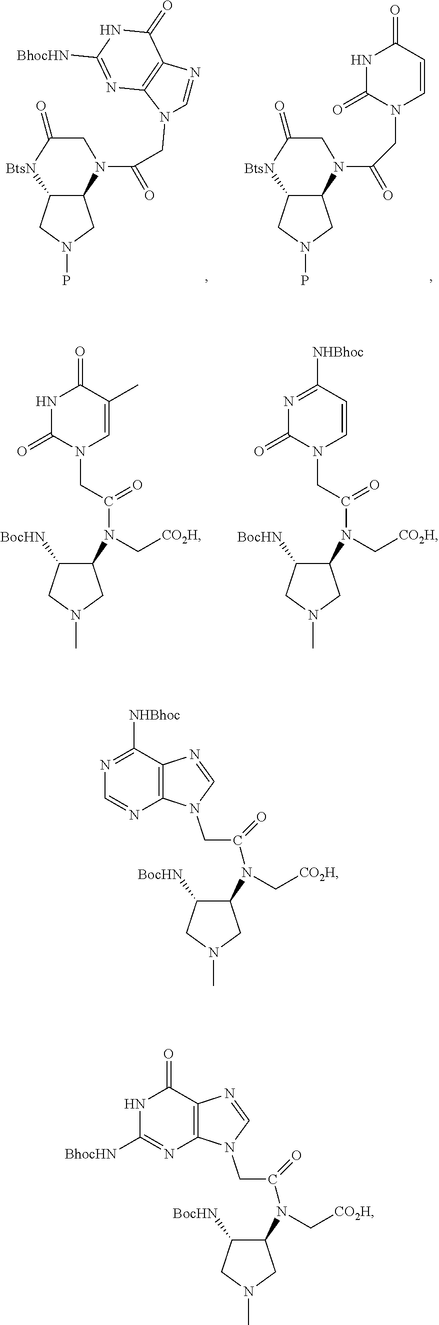

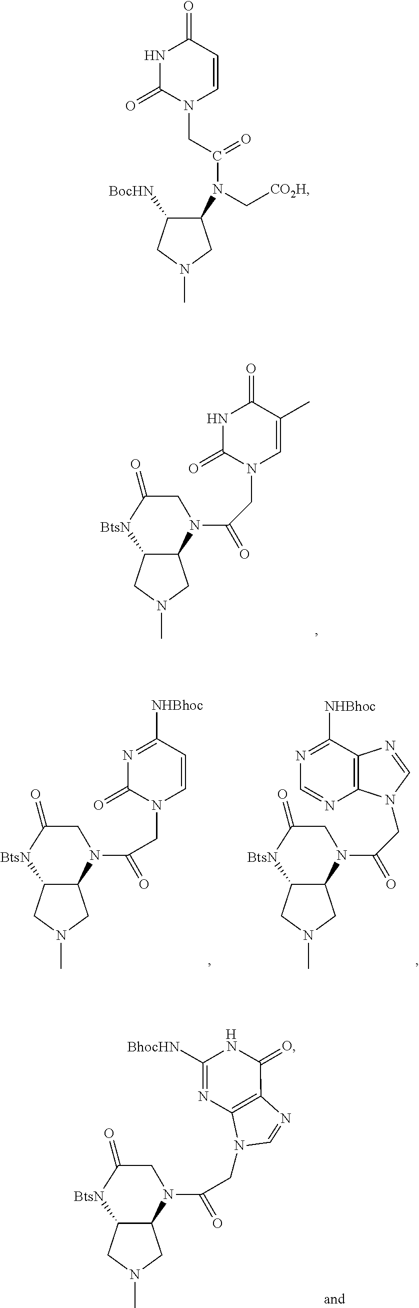

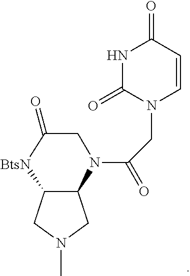

8. A compound according to Formula (II) 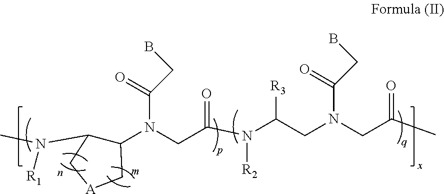
or an optically pure stereoisomer, pharmaceutically acceptable salt, or solvate thereof, wherein
each n and m is an integer independently selected from 0 to 2;
each p, q, and x is an integer independently selected from 0 to 20;
A is selected from the group consisting of —O—, —S—, —NR₄—, and 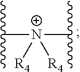
B is selected from the group consisting of 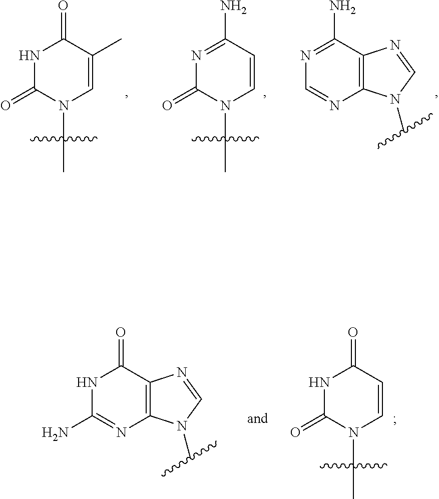
each R₁, R₂ and R₄ is independently H, methyl, ethyl, or propyl; and
R₃ is H, methyl, ethyl, propyl, F, Br, Cl, CF₃, NO₂, OH, OCH₃, CN, amino group unsubstituted or substituted with methyl, ethyl, or propyl, —CH₂(OCH₂CH₂)_{y}OMe, and 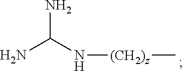
y is an integer selected from 0 to 5; and
z is an integer selected from 1 to 5.

9. The compound of claim 8, wherein n is 1.

10. The compound of claim 8, wherein m is 1.

11. The compound of claim 8, wherein A is —O—.

12. The compound of claim 8, wherein A is

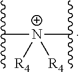

13. The compound of claim 8, wherein R₃ is —CH₂(OCH₂CH₂)_{y}OMe or

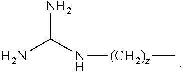

14. A method of improving solubility and/or nucleic acid affinity of a peptide nucleic acid (PNA) comprising: incorporating one or more cyclic structural moieties into a PNA monomer, thereby improving the PNA solubility and/or nucleic acid affinity.

15. The method of claim 14, wherein the one or more cyclic structural moieties comprise tetrahydrofuran or pyrrolidinium moieties.

16. The method of claim 15, wherein the tetrahydrofuran or pyrrolidinium moieties are incorporated into C2-C3 position.

17. A pharmaceutical composition comprising the compound of claim 8 and a pharmaceutically acceptable carrier.

18. A method of reducing expression of a target gene in a cell comprising:
contacting a cell in which the target is expressed with a PNA agent comprising the compound of claim 8,
thereby reducing the expression of the target gene.

19. A method for identifying and/or characterizing PNA agents for target inhibition comprising:
contacting a system in which a target is expressed with a PNA agent comprising the compound of claim 8;
determining a level or activity of the target in the system when the PNA agent is present as compared with a target reference level or activity observed under otherwise comparable conditions when it is absent; and
classifying the PNA agent as a target inhibitor if the level or activity of the target is significantly reduced when the PNA agent is present as compared with the target reference level or activity.

20. A method for treating cancer in a subject comprising administering to the subject the compound of claim 8.

21. The method of claim 20, further comprising administering an anti-cancer treatment.

22. The method of claim 21, wherein the compound is administered prior to, simultaneously with or following the administration of the anti-cancer treatment.

23. The method of claim 20, wherein the compound is administered orally, parenterally, intradermally, transdermally, or by inhalation.
